(19) United States
(12) Patent Application Publication
(10) Pub. No.: US 2024/0058403 A1
(43) Pub. Date: Feb. 22, 2024

(54) YEAST PARTICLES FOR DELIVERY OF WATER-ACTIVATED SELF-EMULSIFYING CANNABINOID FORMULATIONS

(71) Applicant: UNIVERSITY OF MASSACHUSETTS, Boston, MA (US)

(72) Inventors: Gary R. Ostroff, Worcester, MA (US); Ernesto R. Soto, Worcester, MA (US)

(21) Appl. No.: 18/226,834

(22) Filed: Jul. 27, 2023

Related U.S. Application Data
(60) Provisional application No. 63/393,583, filed on Jul. 29, 2022.

(51) Int. Cl. A61K 36/06; A61K 31/00; A61K 9/48; A61K 9/50

(52) U.S. Cl. CPC A61K 36/06; A61K 31/658; A61K 9/4833; A61K 9/5052

(57) ABSTRACT

The present disclosure provides a yeast particle delivery system for controlled release of cannabinoids and other hydrophobic payloads. The disclosure further provides methods of making and methods of using the yeast particle delivery system.

### RELATED APPLICATIONS

The present invention claims the benefit of U.S. Provisional Patent Application Ser. No. 63/393,583, filed Jul. 29, 2022, the content of which is incorporated herein by reference in its entirety for all purposes.

### FIELD OF THE INVENTION

The present invention relates to medicine, pharmacology, and agriculture. More specifically, the invention relates to compositions and delivery systems for hydrophobic payloads.

### BACKGROUND

Cannabinoids are naturally occurring compounds found in the Cannabis sativa plant. Of over 480 different compounds present in the plant, only around 104 are termed cannabinoids. Among these, tetrahydrocannabinol (THC) has received the most attention for being responsible for the intoxicated state sought after by recreational cannabis users. Cannabinoids exert their effects by interacting with specific cannabinoid receptors present on the surface of cells. Cannabinoids produce numerous therapeutic effects. They have antispastic, analgesic, antiemetic, neuroprotective, and anti-inflammatory actions. They are an effective treatment against certain psychiatric diseases. Emerging clinical applications for cannabinoid therapies include Alzheimer's disease, amyotrophic lateral sclerosis (ALS), atherosclerosis, chronic pain, diabetes mellitus, dystonia, epilepsy, fibromyalgia, gastrointestinal disorders, gliomas, cancer, hepatitis C, human immunodeficiency virus (HIV), Huntington's disease, hypertension, incontinence, methicillin-resistant Staphylococcus aureus (MRSA), multiple sclerosis, osteoporosis, posttraumatic stress disorders (PTSD), pruritus, rheumatoid arthritis, sleep apnea and Tourette syndrome.

Currently employed methods of delivery of cannabinoids include delivery by inhalation, ingestion or intraoral delivery methods to the oral mucosa. However, these methods cannot deliver precise doses of cannabinoids in a controlled manner.

Thus, there is a need in the pharmaceutical arts for the development of compositions and methods for delivering cannabinoids to cells and organisms.

### SUMMARY

This disclosure is directed to compositions and methods for controlled cannabinoids delivery.

In one aspect, a yeast particle (YP) delivery system is provided comprising a YP having a hollow internal space; a hydrophobic payload substantially encapsulated within the hollow internal space; and an adjuvant.

In certain exemplary embodiments, the YP delivery system further comprises one or more leave-in solvents encapsulated within the hollow internal space of the YP along with the hydrophobic payload.

In certain exemplary embodiments, the YP delivery system further comprises a release agent encapsulated within the hollow internal space of the YP along with the hydrophobic payload.

In certain exemplary embodiments, the YP delivery system further comprises a sequestering agent encapsulated within the hollow internal space of the YP along with the payload.

In certain exemplary embodiments, the adjuvant comprises a self-emulsifying delivery system (SEDDS) solvent.

In certain exemplary embodiments, the SEDDS solvent comprises lecithin, medium chain triglycerides, glycerin, 1,3-propanediol or a mixture thereof.

In certain exemplary embodiments, the adjuvant further comprises a drying agent.

In certain exemplary embodiments, the leave-in solvent is selected from the group consisting of glycerin, a fatty acid, a surfactant, undecanoic acid, octanoic acid, glycofurol, and any mixture thereof.

In certain exemplary embodiments, the release agent is selected from the group consisting of a glycerin, a fatty acid, a surfactant, undecanoic acid, octanoic acid, glycofurol, PLURONIC® 17R4, IGEPAL CO-520, TRITON™ X-100, Brij58, and Brij35 and any mixtures thereof.

In certain exemplary embodiments, the sequestering agent is selected from the group consisting of a fatty acid, octanoic acid, a fatty acid ester mixture, CAPMUL®, NEOBEE® 1053, Nerolidol, a terpene, a surfactant, TRITON™ X100, BRIJ®, PLURONIC®, a glycerin, a fatty acid, undecanoic acid, octanoic acid, glycofurol, PLURONIC® 17R4, TRITON™ X-100, Brij58, and Brij35 and any mixtures thereof.

In certain exemplary embodiments, the hydrophobic payload is selected from the group consisting of tetrahydrocannabinol (THC), cannabigerol (CBG), cannabidiol (CBD), and cannabinol (CBN), and any mixtures thereof.

In certain exemplary embodiments, the hydrophobic payload further comprises a second distinct hydrophobic substance.

In another aspect, a kit is provided comprising the YP delivery system as described in the aspect and/or embodiments above.

In yet another aspect, a pharmaceutical composition is provided comprising the YP delivery system described in an aspect or an embodiment above and a pharmaceutically acceptable excipient.

In another aspect, a method is provided for delivering a hydrophobic payload to a cell comprising contacting a cell with the YP delivery system or a pharmaceutical composition described in an aspect or an embodiment above.

In yet another aspect, a method is provided for making a YP delivery system comprising the steps of: incubating a yeast particle having a hollow internal space with a hydrophobic payload until the payload becomes substantially enclosed within the hollow internal space; and embedding the YP in an adjuvant and optionally a drying agent.

In certain exemplary embodiments, the method further comprises the step of contacting the YP with a release agent along with the hydrophobic payload.

In certain exemplary embodiments, the method further comprises the step of contacting the YP with a sequestering agent along with the hydrophobic payload.

In another aspect, a method is provided for making a YP delivery system comprising the steps of: incubating a yeast particle (YP) having a hollow internal space with a hydrophobic payload dissolved in a loading solvent until the payload becomes substantially enclosed within the hollow internal space; optionally removing the loading solvent; and embedding the YP in an adjuvant and optionally a drying agent.

In certain exemplary embodiments, the method further comprises the step of contacting the YP with a release agent along with the hydrophobic payload.

In certain exemplary embodiments, the method further comprises the step of contacting the YP with a sequestering agent along with the hydrophobic payload.

In certain exemplary embodiments, the release agent is selected from the group consisting of a glycerin, a fatty acid, a surfactant, undecanoic acid, octanoic acid, glycofurol, PLURONIC® 17R4, IGEPAL CO-520, TRITON™ X-100, Brij58, and Brij35 and any mixtures thereof.

In certain exemplary embodiments, the sequestering agent is selected from the group consisting of a fatty acid, an octanoic acid, a fatty acid ester mixture, CAPMUL®, NEOBEE® 1053, Nerolidol, a terpene, a surfactant, TRITON™ X100, BRIJ®, PLURONIC®, a glycerin, a fatty acid, undecanoic acid, octanoic acid, glycofurol, PLURONIC® 17R4, TRITON™ X-100, Brij58, and Brij35 and any mixtures thereof.

In certain exemplary embodiments, the loading solvent is ethanol, methanol, dimethylsulfoxide (DMSO), or a mixture thereof.

In yet another aspect, a method is provided for delivering a hydrophobic payload to a subject, comprising administering to a subject the YP delivery system as described in embodiments above.

In certain exemplary embodiments, the YP delivery system releases the hydrophobic payload within seconds to about ten minutes after administration.

In certain exemplary embodiments, the YP delivery system is administered to the subject in the nasal cavity.

In certain exemplary embodiments, the YP delivery system is administered to the subject in the buccal cavity.

In certain exemplary embodiments, the YP delivery system releases the hydrophobic payload molecule over a period of about 1 hour to about 24 hours after administration.

### BRIEF DESCRIPTION OF THE DRAWINGS

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1A-FIG. 1B show the chemical structures of cannabinoids (THC: tetrahydrocannabinol, CBD: cannabidiol, CBG: cannabigerol, CBN: cannabinol) and the schematic diagram for loading cannabinoids into GLPs/YPs.

FIG. 2A-FIG. 2B depict light and fluorescent photomicrographs of empty GLPs (control) and tetrahydrocannabinol (THC) loaded GLPs prepared at 1:1 to 5:1 THC:GLP ratios showing encapsulated fluorescent THC-Nile red complex and the average particle size of empty GLP control and GLPs loaded at various THC:GLP weight ratios.

FIG. 3 depicts light and fluorescent photomicrographs of empty GLPs (control) and GLPs co-loaded with THC and leave-in solvent undecanoic acid (UDA) prepared at various ratios of THC:UDA:GLP showing encapsulated fluorescent THC-Nile red complex.

FIG. 4A-FIG. 4B depict light and fluorescent photomicrographs of empty GLPs (control) and cannabidiol (CBD) loaded GLPs prepared at 1:1 to 5:1 CBD:GLP ratios with loading solvent ethanol showing encapsulated fluorescent CBD-Nile red complex and the average particle size of empty GLP control and GLPs loaded at various CBD:GLP weight ratios.

FIG. 5A-FIG. 5B depict light and fluorescent photomicrographs of empty GLPs (control) and cannabidiol (CBD) loaded in GLPs with loading solvent octanoic acid (OA) showing encapsulated fluorescent CBD-Nile red complex and the average particle size of empty and loaded GLPs.

FIG. 6 depicts light and fluorescent photomicrographs of empty GLPs (control) and GLPs co-encapsulating cannabidiol (CBD) and melatonin and leave-in solvents octanoic acid (OA) and glycofurol showing encapsulated fluorescent payload.

FIG. 7 is a schematic diagram showing strategies for in vivo and in vitro release of cannabinoids from YPs/GLPs.

FIG. 8A-FIG. 8B are schematic diagrams showing release of cannabinoids from SEDDS embedded YPs/GLPs loaded with cannabinoids (A) or co-loaded with cannabinoid and a release agent (B).

FIG. 9 shows a flow chart of steps to cannabinoid release assays.

FIG. 10 shows THC released from GLPs (THC:GLP weight ratio 1:1) embedded in different solvents/adjuvants in 1 minute after contact with simulated saliva with or without tissue.

FIG. 11 depicts pie charts showing percentage distribution of THC in different fractions after release from GLPs embedded in SEDDS mixture.

FIG. 12 graphically depicts THC released in 1 minute from GLPs loaded with THC alone or co-loaded with THC and undecanoic acid (UDA) as a release agent. GLPs were embedded in different solvents/adjuvants and THC release was measured after contact with simulated saliva with or without tissue.

FIG. 13 graphically depicts cannabinoids (CBD, CBN, and CBG) released in 1 minute from GLPs loaded with the cannabinoid alone or co-loaded with cannabinoid and octanoic acid (OA) as a release agent.

FIG. 14 is a schematic diagram showing sustained/slow cannabinoid release of payload from YP/GLP samples.

FIG. 15A-FIG. 15B graphically depicts the effect of sequestering agents on sustained release of CBD from GLPs co-loaded with CBD and OA as SA (A) and CBD with various other SAs (B).

FIG. 16 graphically depicts sustained CBD release from GLP-CBD-OA±RAs in simulated saliva with tissue.

FIG. 17 graphically depicts sustained CBD release from GLP-CBD-SA TRITON™ X-100 as RA in simulated saliva+tissue at 37° C.

FIG. 18 graphically depicts sustained THC release from GLP-THC-UDA±RA in simulated saliva+tissue at 37° C.

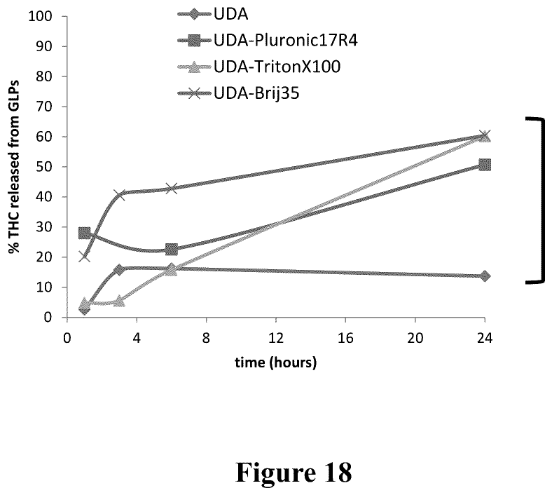

FIG. 19 graphically depicts sustained release of melatonin and CBD (0-4 h) from GLPs in simulated saliva with tissue at 37° C.

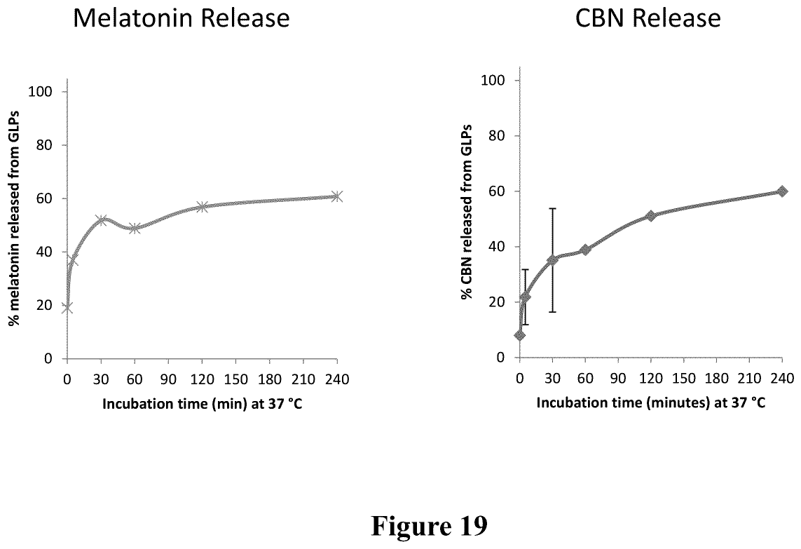

FIG. 20 depicts light and fluorescent photomicrographs of empty GLPs (control) and GLPs encapsulating oleamide payload and leave-in solvents myristic acid (OA) and palmitic acid.

### DETAILED_DESCRIPTION

The present disclosure improves upon conventional encapsulation technologies by providing a yeast particle (YP) delivery system for fast and slow/sustained release of hydrophobic payloads such as cannabinoids.

In the present disclosure, cannabinoids are loaded into the YPs at a payload:YP weight/weight ratio from 1.5:1 up to 5:1 such that the chemical or biologic activities of the payloads are not permanently altered or diminished. The compositions and methods of the present disclosure can achieve increased storage stability and controlled release of the payload from YPs upon contact with water, thereby providing for a significant improvement over existing technologies.

The disclosures of patents, patent applications, and publications in their entireties are hereby incorporated by reference into this application in order to more fully describe the state of the art as known to those skilled therein as of the date of the invention described and claimed herein (e.g., U.S. Pat. Nos. 9,655,360, 10,004,229, European Patent No. 1711058, WO2005070213A2, WO2005113128A1 and associated patents/patent applications). The instant disclosure will govern in the instance that there is any inconsistency between the patents, patent applications, and publications and this disclosure.

That the disclosure may be more readily understood, select terms are defined below.

### Payload Molecules

The yeast particle (YP) delivery system of the present disclosure is useful for in vivo or in vitro delivery of payload molecules to a cell or an organism. This delivery system is useful for the delivery of any hydrophobic, water-insoluble low molecular payloads that cannot be encapsulated at high payload:YP w/w ratios within yeast particles using any art-known method. Any low molecular payload that is a water-insoluble payload is envisioned by the present disclosure.

Cannabinoid Payloads

The term “cannabinoid” in this disclosure refers to any of the diverse chemical compounds that act on cannabinoid receptors on cells in the brain, act on orthosteric or allosteric sites and modulate endocannabinoid activity. Cannabinoids include the phytocannabinoids found in cannabis, hempseed oil, other plants, and synthetic cannabinoids manufactured artificially. They include, but are not limited to, the phytocannabinoids delta-9-tetrahydrocannabinol (THC), cannabidiol (CBD), cannabinol (CBN) cannabigerol (CBG), cannabigerol (CBG), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), or the like; or mixtures or combinations thereof. Other botanical cannabimimetics include N-alkylamides from Echinacea and B-caryophyllene. They include mixtures of phytocannabinoids separated from the plant by extraction techniques and high purity cannabinoids obtained by purification from natural sources or via synthesis.

Terpene Payloads

In certain embodiments, the disclosure provides compositions and methods for the encapsulation and delivery of terpenes payload molecules. Any terpene may be hyperloaded, encapsulated, and delivered according to the methods of the present disclosure. The terpene payload may comprise a single terpene or a mixture of terpenes.

The term “terpene” as used herein refers to terpenes of formula (C₅H₈)ₙ, and terpene derivatives, such as terpene aldehydes. In addition, reference to a single name of a compound will encompass the various isomers of that compound. For example, the term citral includes the cis-isomer citral-a (or geranial) and the trans-isomer citral-b (or neral).

Terpenes are classified as Generally Recognized as Safe (GRAS) and have been used for many years in the flavoring and aroma industries. The list of terpenes which are exempted from US regulations found in EPA regulation 40 C.F.R. Part 152 is incorporated herein by reference in its entirety. Terpenes have a relatively short life span of approximately 28 days once exposed to oxygen (e.g., air). Terpenes decompose to CO₂, further demonstrating the safety and environmental friendliness of the compositions and methods of the disclosure.

Terpenes have been found to inhibit the in vitro growth of bacteria and fungi (Chaumont et al.), Ann. Pharm. Fr., 1992, 50(3): 156-166; Moleyar et al., Int. J. Food Microbiol., 1992, 16(4): 337-342; and Pattnaik et al., Microbios., 1997, 89(358): 39-46) and some internal and external parasites (Hooser et al., J. Am. Vet. Med. Assoc., 1986, 189(8): 905-908). The terpene geraniol is the active component (75%) of rose oil. Rose oil and geraniol at a concentration of 2 mg/L inhibited the in vitro growth of H. pylori. Geraniol was found to inhibit the growth of C. albicans and S. cerevisiae strains by enhancing the rate of potassium leakage and disrupting membrane fluidity (Bard et al., Lipids, 1998, 23(6): 534-538).

There may be different modes of action of terpenes against microorganisms; they (1) interfere with the phospholipid bilayer of the cell membrane, (2) impair a variety of enzyme systems (HMG-reductase), and (3) destroy or inactivate genetic material. It is believed that due to the modes of action of terpenes being so basic, e.g., blocking of cholesterol, that infective agents do not build a resistance to terpenes.

The terpenes, surfactants, and other components of the pre-payloads according to the disclosure may be readily purchased, extracted from natural and genetically engineered cells producing the payload sources, or synthesized using techniques generally known to synthetic chemists. Useful terpenes according to the present disclosure, for safety and regulatory reasons, are at least food grade terpenes, as defined by the United States FDA or equivalent national regulatory body outside the USA. Non-limiting examples of suitable surfactants include sodium lauryl sulphate, polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, polyoxyethylenesorbitan, monooleate, TWEEN®, SPAN® 20, SPAN® 40, SPAN® 60, SPAN® 80, IGEPAL®, TRITON™ X-100, NEOBEE®, Brij 30 or mixtures thereof.

Alternatively, stable terpene solutions can be obtained by mixing terpenes and water at high shear. See PCT Patent Application Publication WO2003/020024. Regardless of how they are prepared, terpenes are prone to oxidation in aqueous emulsion systems, which makes long term storage a problem. Thus, the composition of the present disclosure can comprise an antioxidant to reduce oxidation of the terpene. A non-limiting example of such an anti-oxidant might be rosemary oil, vitamin C, or vitamin E. Preservatives and other additives can also be present such as, for example, antimicrobials, anti-oxidants, chelating agents, and inert gases.

Terpenes can be taken up and stably encapsulated within hollow glucan particles or cell wall particles. See U.S. Pat. No. 9,439,416, the contents of which are incorporated by reference in its entirety. Encapsulation of terpenes into such particles can be achieved by incubation of the particles with the terpene. Nevertheless, terpenes rapidly diffuse from the glucan shell when encapsulated according to conventional methods. Accordingly, certain exemplary embodiments of the present disclosure provide for improved compositions and methods for the encapsulation and delivery of terpenes.

Compositions of the present disclosure can comprise other active compounds, alone or in addition to the terpene component. The compositions can comprise a further active agent in addition to the terpene component, for example, an antimicrobial agent, an anti-fungal agent, an insecticidal agent, an anti-inflammatory agent, an anesthetic, or the like.

Suitable agents include, but are not limited to, antifungals, such as cell wall hydrolases, to the extent they do not degrade the hollow glucan particle or cell wall particle, cell wall synthesis inhibitors, and standard antifungals; antibacterials, such as antiseptics, cell wall hydrolases, synthesis inhibitors, and antibiotics; and insecticides, such as natural insecticides and chitinase.

Antimicrobial Payloads

Certain exemplary embodiments of the present disclosure provide for compositions and methods for the loading and delivery of hydrophobic payload molecules with antimicrobial activity effective against classes of organisms such as Gram-positive bacteria, Gram-negative bacteria, fungi, viruses, pests, nematodes and insects.

As used herein, the term “antimicrobial” refers to the ability of a compound to inhibit or irreversibly prevent the growth of a microorganism. Such inhibition or prevention can be through a microbicidal action or microbistatic inhibition. The term “microbicidal inhibition” refers to the ability of the antimicrobial compound to kill, or irrevocably damage the target organism. The term “microbiostatic inhibition” as used herein refers to the ability of the antimicrobial compound to inhibit the growth of the target organism without death.

A compound with microbicidal or microbistatic inhibitory properties can be applied to an environment either presently exhibiting microbial growth (i.e., therapeutic treatment) or to an environment at risk of supporting such growth (i.e., prevention or prophylaxis). An environment capable of sustaining microbial growth refers to a fluid, substance, or organism where microbial growth can occur or where microbes can exist. Such environments can be, for example, animal tissue or bodily fluids, water and other liquids, food, food products or food extracts, crops, and certain inanimate objects. It is not necessary that the environment promotes the growth of the microbe, only that it permits its subsistence.

Any suitable hydrophobic antimicrobial compound may be encapsulated according to the methods presently described. In certain nonlimiting embodiments, the antimicrobial compound is an antibiotic, such as aminoglycosides (gentamycin, kanamycin), macrolides (erythromycin), rifamycins, novobiocin, fusidic acid, cationic and zwitterionic peptides, cycloserine, and rifampicin. The hydrophobic antimicrobial payload component may comprise a single microbial or a mixture of antimicrobials.

Chemotherapeutic Payloads

Certain exemplary embodiments of the present disclosure also provide compositions and methods for the encapsulation and delivery of hydrophobic payload molecules with chemotherapeutic or anticancer properties. Any solid or hematological cancer may be treated with the hydrophobic payload molecules presently disclosed.

Exemplary useful chemotherapeutic agents include alkylating agents, anti-metabolites, alkaloids, and miscellaneous agents (including hormones), and certain antibiotics. For example, anthracyclines are one of the more commonly used chemotherapeutic antibiotics. Anthracycline antibiotics are produced by the microorganism Streptomyces peuceitius var. caesius. Anthracycline antibiotics have tetracycline ring structures with an unusual sugar, daunosamine, attached by glycosidic linkage. Cytotoxic agents of this class all have quinone and hydroquinone moieties on adjacent rings that permit them to function as electron-accepting and donating agents.

Anthracyclines achieve their cytotoxic effect by several mechanisms, including intercalation between DNA strands, thereby interfering with DNA and RNA synthesis; production of free radicals that react with and damage intracellular proteins and nucleic acids; chelation of divalent cations; and reaction with cell membranes. The wide range of potential sites of action may account for the broad efficacy as well as the toxicity of the anthracyclines.

Any suitable hydrophobic chemotherapeutic or antitumor compound may be encapsulated according to the methods presently described. In certain embodiments, the chemotherapeutic or antitumor compound is selected from the group consisting of doxorubicin, epirubicin, idarubicin, and mitoxantrone. The chemotherapeutic or anticancer payload component may comprise a single payload molecule or a mixture of payload molecules.

Non-Steroidal Anti-Inflammatory Drug (NSAID) Payloads

The disclosure also provides compositions and methods for the encapsulation and delivery of payload molecules with analgesic and anti-inflammatory properties. The analgesic or anti-inflammatory payload component may comprise a single pro-payload molecule or a mixture of payload molecules. Any useful analgesic or anti-inflammatory compound may be encapsulated according to the methods presently described.

In certain embodiments, the analgesic or anti-inflammatory compound is selected from the group consisting of aspirin, acetaminophen, d-propoxyphene, fenoprofen, flurbiprofen, ibuprofen, ketoprofen, naproxen, and oxaprozin.

Nonsteroidal anti-inflammatory drugs (NSAIDs) are a drug class that reduce pain, decrease fever, prevent blood clots and, in higher doses, decrease inflammation. Useful NSAIDs include, without limitation, aspirin, ibuprofen and naproxen.

Naproxen is a well-known NSAID, with a daily dose ranging from about 250 to about 1500 milligrams, or from about 500 to about 1000 milligrams. Naproxen, and other analgesic drugs, can be administered in multiple doses over 12 or 24 hours.

Additionally, a higher initial dose, followed by relatively low maintenance doses, can be delivered. See, e.g., Palmisano et al., Advances in Therapy, Vol. 5, No. 4, July/August 1988; describing the use of multiple doses of ketoprofen (initial dose of 150 mg followed by subsequent doses of 75 mg) and ibuprofen (initial dose of 800 mg followed by subsequent doses of 400 mg).

Controlled release pharmaceutical dosage forms can be used to optimize drug delivery and enhance patient compliance. A pharmaceutical dosage form can deliver more than one drug, each at a modified rate.

### Yeast Particles

As used herein, a “yeast particle” or “YP” refers to readily available, biodegradable, substantially spherical, hollow particles with an intact shell of about 2-4 μm in diameter. YPs may be obtained as a byproduct of some food grade Baker's yeast (i.e., Saccharomyces cerevisiae) extract manufacturing processes. YPs include, extracted yeast cell wall particles (YCWPs), yeast cell particles (YCPs), glucan particles (GPs), yeast glucan particles (YGPs), yeast glucan-mannan particle (YGMP), glucan lipid particles (GLPs), whole glucan particles (WGPs) and the like.

YPs comprise an intact shell composed of remnants of cell wall components after yeast cell extraction. YPs retain the three-dimensional structure of the yeast cell from which they are derived and thus, upon extraction of cellular components of a yeast cell leaves behind hollow particles which have a large cavity or “hollow inner space” enclosed within the yeast cell wall. The hollow inner space is suitable for encapsulating many payloads. The yeast walls may suitably be derived from, inter alia, Baker's yeast cells (available from Sigma Chemical Corp., St. Louis, MO). Yeast cell wall particles with desirable properties can also be obtained from Biorigin (Sao Paulo, Brazil).

Methods of preparing extracted yeast cell wall particles are known in the art, and are described, for example in U.S. Pat. Nos. 4,992,540, 5,082,936, 5,028,703, 5,032,401, 5,322,841, 5,401,727, 5,504,079, 5,968,811, 6,444,448, 6,476,003, published U.S. applications 2003/0216346 A1, 2004/0014715 A1, and PCT published application WO 02/12348 A2, which are specifically incorporated herein by reference.

Alternative particles are those known by the trade names SAF-Mannan (SAF Agri, 5 Minneapolis, MN) and Nutrex (Sensient Technologies, Milwaukee, WI) and many other yeast cell product producers. These are substantially intact hollow glucan particles that are the insoluble waste stream from the yeast extract manufacturing process. During the production of yeast extracts the soluble components of partially autolysed yeast cells are removed and the insoluble residue contains YPs. These substantially hollow glucan particles comprise approximately 25-35% beta 1,3-glucan w/w. A key attribute of these materials are that they may contain more than 10% lipid w/w and are very effective at absorbing hydrophobic payloads. In addition, as a waste stream product, they are a relatively cheap source of substantially intact hollow glucan particles.

### Pre-Treatment of Yeast Particles

Pre-treatment of YPs with water or appropriate solvent(s), such as dimethyl sulfoxide or alcohol, is needed for efficient encapsulation and release of payloads. For example, encapsulation of limonene powder does not work unless some water is present (Errenst et al. Encapsulation of limonene in yeast cells using the concentrated powder form technology. J. Supercrit. Fluid 2021, 168, 105076). Dardelle et al. demonstrated that a minimum of 20% hydration is necessary for limonene release (Dardelle et al. Flavour-Encapsulation and flavour-release performances of a commercial yeast-based delivery system. Food Hydrocoll. 2007, 21, 953-960.). Dimopoulous et al. also highlighted the need for water activity (aw)>0.7 to obtain release (Dimopoulos et al. Cell permeabilization processes for improved encapsulation of oregano essential oil in yeast cells. J. Food Eng. 2021, 294, 110408).

Dry YPs can be hydrated by incubation with a variety of aqueous solutions. Suitable aqueous solutions include, but are not limited to: water; saline, e.g., phosphate buffered saline; any buffer solution known in the art with a pH between 3 and 11; any acid solution known in the art with a pH ≥1.5; any basic solution known in the art with a pH <11; any salt solution known in the art that does not chemically interfere with the payload, and the like.

### Payload:YP Ratio

YPs used herein may encapsulate payloads with a capacity of greater than <2:1 payload:YP weight ratio. In certain embodiments, the weight by weight (w/w) ratio of payload:YP can range from about 1:1 to about 10:1, from about 1.5:1 to about 7.5:1, or from about 2.5:1 to about 5:1. For example, the ratio of payload:YP can be about 1.0:1, about 1.1:1, about 1.2:1, about 1.3:1, about 1.4:1, about 1.5:1, about 1.6:1, about 1.7:1, about 1.8:1, about 1.9:1, about 2.1:1, about 2.2:1, about 2.3:1, about 2.4:1, about 2.5:1, about 2.6:1, about 2.7:1, about 2.8:1, about 2.9:1, about 3.1:1, 3.2:1, 3.3:1, about 3.4:1, 3.5:1, 3.6:1, 3.7:1, 3.8:1, 3.9:1, about 4.1:1, about 4.2:1, about 4.3:1, about 4.4:1, about 4.5:1, about 4.6:1, about 4.7:1, about 4.8:1, about 4.9:1, about 5.1:1, about 5.2:1, about 5.3:1, about 5.4:1, about 5.5:1, about 5.6:1, about 5.7:1, about 5.8:1, about 5.9:1, about 6.1:1, 6.2:1, 6.3:1, about 6.4:1, about 6.5:1, about 6.6:1, about 6.7:1, about 6.8:1, about 6.9:1, about 7.1:1, about 7.2:1, about 7.3:1, about 7.4:1, about 7.5:1, about 7.6:1, about 7.7:1, about 7.8:1, about 7.9:1, about 8.1:1, about 8.2:1, about 8.3:1, about 8.4:1, about 8.5:1, about 8.6:1, about 8.7:1, about 8.8:1, about 8.9:1, about 9.1:1, about 9.2:1, about 9.3:1, about 9.4:1, about 9.5:1, about 9.6:1, about 9.7:1, about 9.8:1, about 9.9:1 or about 10:1.

### Payload Encapsulation Efficiency and Encapsulation Yield

Two parameters used to evaluate the payload encapsulation process are encapsulation yield (EY) and encapsulation efficiency (EE). EY, expressed as a percent value, is the weight ratio between payload and YP. EE, expressed as a percent value, is the weight ratio of the payload loaded or encapsulated in a YP with respect to the payload's initial mass. High EE is desirable to maximize payload delivery. In certain embodiments, EE can range from about 50% to about 100%. For example, the payload encapsulation efficiency can be about 50%, about 55%, about 65%, about 70%, about 75%, about 80%, about 90%, about 91%, about 92%, about 93%, about 94%, about 95%, about 96%, about 97%, about 98%, about 99%, or about 100%.

In certain embodiments, at least one payload of a YP is substantially encapsulated in a YP. In certain embodiments, a payload is “substantially encapsulated” by a YP if it is about 50% to about 100% encapsulated. For example, the payload can be about 50%, about 55%, about 65%, about 70%, about 75%, about 80%, about 90%, about 91%, about 92%, about 93%, about 94%, about 95%, about 96%, about 97%, about 98%, about 99%, or about 100% encapsulated in a YP.

### Solvents, Co-Solvents, Sequestering Agents and Release Agents

Solvents may be added during the encapsulation process to facilitate loading of payloads in the YPs. Certain payloads of the present disclosure are water-insoluble or have low water solubility and may be loaded into YPs with a solvent that is compatible with yeast particles. In certain nonlimiting embodiments, the solvent may be an organic solvent. Suitable solvents include, but are not limited to, be acetone, dichloromethane, ethyl acetate, alcohols such as ethanol or methanol, dimethyl sulfoxide (DMSO), methanol-chloroform, geraniol, eugenol, octanoic acid, undecanoic acid, lauric acid, oleic acid, myristic acid, palmitic acid, hexane, petroleum ether, toluene, NEOBEE® and the like. After a payload is completely encapsulated, the yeast particle and payloads may be processed to remove the solvent from the YP-payload formulation. Organic solvents such as acetone, dichloromethane, ethyl acetate, methanol, and DMSO may be unsafe for human administration and should be removed after a payload is completely encapsulated. Alternatively, the solvent used to facilitate payload encapsulation may be safe for human administration and can be left inside the YP along with the hydrophobic payload as a “leave-in solvent.”

Suitable leave-in solvents include, but are not limited to, geraniol (G), eugenol (E), thymol (T) or mixture thereof (G+E+T), N,N-dimethyl-decanamide (DMDA; for delivery of agricultural payloads), glycofurol (for delivery of pharmaceutical payloads), octanoic acid, octanoic acid, undecanoic acid, lauric acid, oleic acid, myristic acid, palmitic acid, or a mixture thereof.

Certain leave-in solvents may serve as “sequestering agents” by delaying the release of the payload from YPs.

Suitable sequestering agents include fatty acids, octanoic acid, a fatty acid ester mixture, CAPMUL®, NEOBEE® 1053, Nerolidol, a terpene, a surfactant, TRITON™ X100, BRIJ®, PLURONIC®, a glycerin, a fatty acid, undecanoic acid, octanoic acid, PLURONIC® 17R4, TRITON™ X-100, Brij58, and Brij35 or any mixtures thereof.

Certain leave-in solvents may serve as “release agents” by accelerating the release of the payload from YPs.

Suitable release agents include glycerin, fatty acids such as undecanoic acid or octanoic acid, a surfactant, undecanoic acid, octanoic acid, glycofurol, PLURONIC® 17R4, IGEPAL CO-520, TRITON™ X-100, Brij58, and Brij35 or any mixtures thereof.

Use of certain sequestering and/or release agents allows for a fast release of payload, that is payload release from YPs within about 10 minutes; while use of certain sequestering and/or release agents allows sustained release of payload, that payload release from YPs over a period of about 1 hour to about 24 hours.

The term “medium chain triglyceride” (MCT) as used in the present disclosure refers to a class of triglyceride oil that are typically naturally derived from fatty acids that are usually about 8 to about 12 carbons in length.

Suitable medium chain triglycerides include caproic acid, hexanoic acid, caprylic acid, octanoic acid, capric acid, decanoic acid, lauric acid or dodecanoic acid.

### Surfactants

The term “surfactant” in this disclosure refers to compounds that lower the surface tension (or interfacial tension) between two liquids or between a liquid and a solid act as emulsifiers, dispersants, wetting agents and viscosity modifiers. In one embodiment surfactants means amphiphilic molecules which are manufactured by chemical processes or purified from natural sources or processes that can be anionic, cationic, nonionic, and zwitterionic. In one embodiment, “surfactant” means any molecule having both a hydrophilic group (e.g., a polar group), which energetically prefers solvation by water, and a hydrophobic group which is not well solvated by water. The term “nonionic surfactant” is a known term in the art and generally refers to a surfactant molecule whose hydrophilic group (e.g., polar group) is not electrostatically charged.

Surfactants are generally low to moderate weight compounds which contain a hydrophobic portion, which is generally readily soluble in oil, but sparingly soluble or insoluble in water, and a hydrophilic portion, which is sparingly soluble or insoluble in oil, but readily soluble in water. In addition to protecting against growth and aggregation and stabilizing the organic compound delivery vehicle, surfactants are also useful as excipients in organic compound delivery systems and formulations because they increase the effective solubility of an otherwise poorly soluble or non-soluble organic compound, and may decrease hydrolytic degradation, decrease toxicity and generally improve bioavailability. Surfactants may also provide selected and advantageous effects on drug release rate and selectivity of drug uptake. Surfactants are generally classified as either anionic, cationic, zwiterionic, or nonionic.

Suitable surfactants include, but are not limited to, sodium lauryl sulphate, polysorbate 20, polysorbate 80, polysorbate 40, polysorbate 60, polyglyceryl ester, mono fatty acid ester of polyoxyethylene sorbitan, polyglyceryl monooleate, decaglyceryl monocaprylate, propylene glycol dicaprilate, polyethylenepolypropylene glycol, triglycerol monostearate, polyoxyethylenesorbitan, monooleate, TWEEN®, SPAN® 20, SPAN® 40, SPAN® 60, Span® 80, IGEPAL®, TRITON™ X-100, LABRASOL®, CREMPHOR® EL, GELUCIRE® 50/13, CREMPHOR® RH 40, GELUCIRE® 44/14, NEOBEE® Brij 30 and the like, and any mixtures thereof.

### Self-Emulsifying Drug Delivery System (SEDDS)

A self-emulsifying drug delivery system (SEDDS) provides for water-activated payload release. SEDDS can enhance delivery and bioavailability of hydrophobic payloads with poor solubility and hydrophilic payloads with lower permeability. SEDDSs are thermodynamically stable formulations that are comprised of an isotropic mixture of oils, co-surfactants, surfactants, solvents and co-solvents. Upon slight stirring or exposure to aqueous media, a SEDDS forms an oil in water emulsion or micelles with submicron droplet size. SEDDSs self-emulsify quickly in the aqueous environment of the gastrointestinal tract. The payloads of the present disclosure may be encapsulated in YPs as part of a SEDDS mixture. When YPs in SEDDS mixture comes in contact with bodily fluids, e.g., via saliva, mucus, gastric fluid, nasal fluid, buccal fluid, oral fluid, the payload is released from YPs into micelles containing payload. A micelle may have a diameter ranging from about 5 nm to about 10 μm. For example, in some embodiments, the micelle size ranges from about 5 nm to about 50 nm, from about 50 nm to about 100 nm, from about 100 nm to about 200 nm, or from about 150 nm to about 350 nm.

A SEDDS has surfactants as one of its components. Non-ionic surfactants are commonly used and their concentrations range from about 5%-10% w/w, about 10-20% w/w, about 20-30% w/w, about 30-40% w/w, about 40-50% w/w, about 50-60% w/w. In certain embodiments concentration of the surfactant is about 5%, about 7.5%, about 10%, about 15%, about 20%, about 25%, about 30%, about 35%, about 40%, about 45%, about 50%, about 55%, about 60%. SEDDS usually require a non-ionic surfactants with hydrophilic-lipophilic balance (HLB) value of greater than 12 for the formation of oil-in-water dispersion with a droplet size below 100 nm spontaneously upon dilution in the gastrointestinal tract. The surfactant which possesses good drug solubilizing property may not exhibit good oil affinity. The stable emulsion formation upon dilution is achieved by the right blend of high and low HLB surfactants. When surfactant or cosurfactant contribute to solubilization of the drug, the drug precipitation could be a risk as there could be lowering of the solvent capacity of surfactant or cosurfactant because of dilution of emulsion in the gastrointestinal tract. Thus, the co-surfactant and surfactant have to be carefully chosen based on their oil emulsification properties than their drug solubilizing ability. Usually, alcohols of medium- to short-chain length (C3-C8) are used as co-surfactants, which tend to increase the fluidity of the interface and reduce the interfacial tension. Commonly used co-surfactants are ethanol, isopropyl alcohol, 1-butanol, propylene glycol, and transcutol HP.

### Temperature Stabilizing Agents

The storage stability of YPs containing payloads may be improved by addition of one or more temperature stabilizing agents. Common temperature stabilizing agents include sugars such sucrose, trehalose, glycerol, or sorbitol. Disaccharides such as sucrose and trehalose are natural cryoprotectants with good protective properties. A temperature stabilizing agent may comprise a carbohydrate component including between about 10% and 80% oligosaccharide, between about 5% and 30% disaccharide or between about 1% and 10% polysaccharide, and a protein component including between about 0.5% and 40% protein, e.g., hydrolyzed animal or plant proteins, based on the total weight of the composition. Ascorbic acid ions may be used in some embodiments for stabilization at higher temperature and humidity exposure. Alternatively, a combination of citrate and/or ascorbate ions with protein or protein hydrolysate may be used. In certain nonlimiting embodiments, the temperature stabilizing agent may be a glycerin. In certain nonlimiting embodiment temperature stabilizing agent may be glycerin at a concentration of about 5%, about 10%, about 15%, about 20%, about 25%, about 30%, about 35%, about 40%, about 45% or about 50%. In certain nonlimiting embodiment temperature stabilizing agent may be glycerin at a concentration of 1-5%, 5-10%, 10-15%, 15-20%, 20-25%, 25-30%, 35-40%, 40-45%, or 45%-50%.

### Micelles

As used herein, “micelle” refers to aggregates formed by surfactants that typically form when a surfactant is present in an aqueous composition, typically when the surfactant is used at a concentration above the critical micelle concentration (CMC). In micelles, the hydrophilic portions of the surfactant molecules contact the aqueous or the water phase, while the hydrophobic portions form the core of the micelle, which can encapsulate non-polar ingredient(s), for example, a cannabinoid. Typically, the surfactants form micelles containing the non-polar ingredient at their center. Transmission electron microscopy (TEM) and dynamic light scattering (DLS) are commonly used to show the overall morphology and size of micelles.

Surfactant micelles are used as adjuvants and drug carrier systems in many areas of pharmaceutical technology. Micelles have been used to increase bioavailability or decrease adverse effects of the drugs. The small size of micelles plays a key role in transport across membranes including the blood brain barrier. An increase in micelle size generally lowers the in vivo performance of the encapsulated drug.

### Articles of Manufacture, Compositions, and Methods

In another aspect, the present disclosure provides an article of manufacture or kit comprising a first container containing a hydrophobic payload molecule, wherein the payload molecule is selected from the group consisting of a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, a second container containing a yeast particle delivery system comprising a yeast cell wall particle, and instructions for use.

In another aspect, the present disclosure provides methods of making a yeast particle delivery system comprising the steps of providing an extracted yeast cell wall comprising beta-glucan, the intact yeast cell wall defining an internal space; incubating the hydrophobic payload with the yeast particle in the presence or absence of a solvent, wherein the hydrophobic payload molecule becomes enclosed within the internal space, thereby forming the yeast particle delivery system.

In another aspect, the present disclosure provides a pharmaceutical composition comprising a yeast particle delivery system comprising a yeast cell wall particle, a hydrophobic payload molecule, wherein the payload molecule is selected from the group consisting of a polynucleotide, a polynucleotide, a peptide, a protein, a small organic active agent, a small inorganic active agent, a microbicide, a fungicide, an insecticide, a nematocide, a pesticide, an antibiotic, an analgesic, a non-steroidal anti-inflammatory drug (NSAID), a terpene, a terpenoid, a tetrahydrocannabinol, a cannabidiol, a chemotherapeutic, a dietary supplement, and mixtures thereof, and a pharmaceutically acceptable excipient.

In another aspect, the present disclosure provides methods of using the yeast particle delivery system. In certain embodiments, the disclosure provides a method of delivering a payload molecule of the present disclosure to a cell, comprising: (a) incubating a hydrophobic payload molecule with a yeast cell wall particle defining an internal space and comprising beta glucan, wherein the payload molecule becomes at least partially enclosed within the internal space, thereby forming a yeast particle delivery system; and (b) contacting a cell with the particulate delivery system under conditions that permit internalization of the particulate delivery system and release and delivery of the payload molecule within the cell.

### Agricultural and Industrial Compositions and Methods

The compositions and methods of the present disclosure are useful in the fields of consumer and industrial products, e.g., in food, human and animal drugs, cosmetics, and agriculture. In some embodiments, the compositions and methods of the present disclosure extend to agricultural applications. In certain embodiments, the present disclosure relates to the development and delivery of stable and controlled-release microbiocides, fungicides, insecticides, nematocides, and pesticides to agricultural species, e.g., plants and/or animals. In certain exemplary embodiments, the present disclosure relates to the development and delivery of stable and controlled-release cannabinoids to agricultural species, e.g., animals such as livestock or pets.

In some embodiments of the present disclosure, any of the compositions described above may be formulated in a deliverable form suited to a particular application. Deliverable forms that can be used in accordance with embodiments of the present disclosure include, but are not limited to, liquids, emulsions, emulsifiable concentrates, solids, aqueous suspensions, oily dispersions, pastes, granules, powders, dusts, fumigants, and aerosol sprays. Suitable deliverable forms can be selected and formulated by those skilled in the art using methods currently known in the art. The compositions can be provided in combination with an agriculturally, food, or pharmaceutically acceptable carrier or excipient in a liquid, solid, or gel-like form. For solid compositions, suitable carriers include pharmaceutical or food grades of mannitol, lactose, starch, magnesium stearate, sodium saccharin, talc, cellulose, glucose, sucrose, and magnesium carbonate. Suitably, the formulation is in tablet or pellet form. As suitable carrier could also be a human or animal food material. Additionally, conventional agricultural carriers could also be used.

Cannabinoids are useful for treatment of chronic pain, cancer, chemotherapy-induced nausea and vomiting, anorexia and weight loss associated with HIV, irritable bowel syndrome, epilepsy, spasticity, Tourette syndrome, amyotrophic lateral sclerosis, Huntington's disease, Parkinson's disease, dystonia, dementia, glaucoma, traumatic brain injury, addiction, anxiety, depression, sleep disorders, posttraumatic stress disorder, and schizophrenia and other psychoses. The National Institutes of Health (NIH) reports 162 clinical trials on cannabis-based drugs. In 2018, the medical cannabis market was estimated at about 13.4 billion USD and is expected to reach 66.3 billion USD by 2025. The present disclosure provides a novel delivery system for controlled release of cannabinoids and methods of use.

### Pharmaceutical Compositions and Administration

In addition, the compositions and methods of the present disclosure are useful in the fields of industrial and consumer products and medicines, e.g., in food, human and animal drugs, and cosmetics, and the like. In some embodiments, this disclosure provides for compositions and methods for use in both human and veterinary medicine. In certain embodiments, the present disclosure relates to therapeutic treatment of mammals, birds, and fish. For example, the compositions and methods of the present disclosure are useful for therapeutic treatment of mammalian species including, but not limited to, human, non-human primate, bovine, ovine, porcine, equine, canine, and feline species.

The routes of administration of the delivery system include but are not limited to oral, buccal, sublingual, pulmonary, transdermal, transmucosal, as well as subcutaneous, intraperitoneal, intravenous, and intramuscular injection. Exemplary routes of administration are oral, buccal, sublingual, pulmonary, and transmucosal.

The yeast particle delivery system of the present disclosure is administered to a patient in a therapeutically effective amount. The yeast particle delivery system can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using a controlled release formulation. It is also noted that the dose of the compound can be varied over time. The particulate delivery system can be administered using an immediate release formulation, or using a controlled release formulation, or combinations thereof. The term “controlled release” includes sustained release, delayed release, and combinations thereof, as well as release mediated by chemical (e.g., pH) and/or biological (e.g., enzyme) hydrolysis.

A pharmaceutical composition of the disclosure can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the animal or human treated, and further depending upon the route by which the composition is to be administered. By way of example, the composition can comprise between 0.1% and 100% (w/w) active ingredient. For example, the active ingredient weight in the pharmaceutical composition may be at least about 1%, 5%, 10%, 15%, 20%, 25%, 30%, 35%, 40%, 45%, 50%, 55%, 60%, 65%, 70%, 75%, 80%, 85%, 90%, 95%, or 100%. A unit dose of a pharmaceutical composition of the disclosure will generally comprise from about 100 milligrams to about 2 grams of the active ingredient, or from about 200 milligrams to about 1.0 gram of the active ingredient.

In addition, the yeast particle delivery system of the present disclosure can be administered alone, in combination with a yeast particle delivery system with a different payload, or with other pharmaceutically active compounds. The other pharmaceutically active compounds can be selected to treat the same condition as the yeast particle delivery system or a different condition.

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the disclosure relates to a kit comprising a pharmaceutical composition of the disclosure and instructional material. Instructional material includes a publication, a recording, a diagram, or any other medium of expression which is used to communicate the usefulness of the pharmaceutical composition of the disclosure for one of the purposes set forth herein in a human. The instructional material can also, for example, describe an appropriate dose of the pharmaceutical composition of the disclosure. The instructional material of the kit of the disclosure can, for example, be affixed to a container which contains a pharmaceutical composition of the disclosure or be shipped together with a container which contains the pharmaceutical composition. Alternatively, the instructional material can be shipped separately from the container with the intention that the instructional material and the pharmaceutical composition be used cooperatively by the recipient.

The disclosure also includes a kit comprising a pharmaceutical composition of the disclosure and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein.

For example, a kit may comprise two separate pharmaceutical compositions comprising respectively a first composition comprising a particulate delivery system and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include, without limitation, syringes, boxes, and bags. Typically, a kit comprises directions for the administration of the separate components. The kit form is advantageous when the separate components are administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms, e.g., tablets and capsules. Blister packs generally consist of a sheet of relatively stiff material covered with a foil of, e.g., a transparent plastic material. During the packaging process, recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . Second Week, Monday, Tuesday,” etc. Other variations of memory aids will be readily apparent. Dosing can be hourly, e.g., every hour, every two hours, every four hours, every eight hours etc.). Dosing can be weekly, biweekly, every four weeks, etc. A “daily dose” can be a single tablet or capsule or several pills or capsules to be taken on a given day. Also, a daily dose of a particulate delivery system composition can consist of one tablet or capsule, while a daily dose of the second compound can consist of several tablets or capsules and vice versa. The memory aid should reflect this and assist in correct administration.

In another embodiment of the present disclosure, a dispenser designed to dispense the daily doses one at a time in the order of their intended use is provided. The dispenser may be equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A yeast particle delivery system composition, optionally comprising other pharmaceutically active compounds, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound. Parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols, e.g., propylene glycol, polyethylene glycol, and glycerol, and suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, chewing gums, strips for sub-lingual delivery, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (e.g., powder or granular) form for reconstitution with a suitable vehicle (e.g., sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, and sorbic acid. It may also be desirable to include isotonic agents, for example, sugars, and sodium chloride. Prolonged absorption of injectable pharmaceutical compositions can be brought about by the use of agents capable of delaying absorption, for example, aluminum monostearate and/or gelatin.

Dosage forms can include solid or injectable implants or depots. In certain embodiments, the implant comprises an aliquot of the particulate delivery system and a biodegradable polymer. In certain embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester), and a polyphosphazene.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the particulate delivery system can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the particulate delivery system, e.g. in the region of the Peyer's patches in the small intestine. By way of example, a material such as glyceryl monostearate or glyceryl distearate can be used to coat tablets. Further by way of example, tablets can be coated using methods described in U.S. Pat. Nos. 4,256,108; 4,160,452; and U.S. Pat. No. 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the particulate delivery system in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols. Hard capsules comprising the particulate delivery system can be made using a physiologically degradable composition, such as gelatin. Such hard capsules comprise the particulate delivery system, and can further comprise additional ingredients including, for example, an inert solid diluent such as calcium carbonate, calcium phosphate, or kaolin. Soft gelatin capsules comprising the particulate delivery system can be made using a physiologically degradable composition, such as gelatin. Such soft capsules comprise the particulate delivery system, which can be mixed with water or an oil medium such as peanut oil, liquid paraffin, or olive oil.

Buccal compositions can be made using known technology. Buccal compositions administered in the buccal cavity specifically release agents in the buccal cavity or when swallowed, in the gastrointestinal tract particularly small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that may alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, e.g., almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropyl methylcellulose, aluminum metahydroxide, bentonite, or mixtures of these substances. Liquid formulations of a pharmaceutical composition of the disclosure that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

For topical administration liquids, suspension, lotions, creams, gels, ointments, drops, suppositories, sprays and powders may be used. Conventional pharmaceutical carriers, aqueous, powder or oily bases, and thickeners can be used as necessary or desirable.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The particulate delivery systems described herein are not exposed to high cooking temperatures for extended periods of time, in order to minimize degradation of the compounds.

Compositions for rectal or vaginal administration can be prepared by mixing a particulate delivery system with suitable non-irritating excipients or carriers such as cocoa butter, polyethylene glycol or a suppository wax, which are solid at ordinary room temperature, but liquid at body temperature, and therefore, melt in the rectum or vaginal cavity and release the particulate delivery system. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the disclosure can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F. at atmospheric pressure. Generally, the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent, e.g., having a particle size of the same order as particles comprising the particulate delivery system.

Pharmaceutical compositions of the disclosure formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface-active agent, or a preservative such as methylhydroxybenzoate.

The formulations described herein as being useful for pulmonary delivery are also useful for intranasal delivery of a pharmaceutical composition of the disclosure. Another formulation suitable for intranasal administration is a coarse powder comprising the particulate delivery system. Such a formulation is administered in the manner in which snuff is taken, i.e., by rapid inhalation through the nasal passage from a container of the powder held close to the nares.

A pharmaceutical composition of the disclosure can be prepared, packaged, or sold in a formulation suitable for buccal administration. Such formulations can, for example, be in the form of tablets, lozenges or meltwaway strips made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

Reference will now be made to specific examples illustrating the disclosure. It is to be understood that the examples are provided to illustrate exemplary embodiments and that no limitation to the scope of the disclosure is intended thereby.

### EXAMPLES

### Example 1: Production of Yeast Particles

YPs of this example were 3-5 μm intact hollow and porous microparticles derived from Baker's yeast and were composed primarily of 1→6-β branched, 1→3-β-glucan, chitin and mannan.

Preparation of extracted yeast cell wall particles is known in the art, and is described, for example in U.S. Pat. Nos. 4,992,540, 5,082,936, 5,028,703, 5,032,401, 5,322,841, 5,401,727, 5,504,079, 5,968,811, 6,444,448 B1, 6,476,003 B1, published U.S. applications 2003/0216346 A1, 2004/0014715 A1, and published PCT application WO 02/12348 A2, the teachings of which are incorporated herein by reference.

A form of extracted yeast cell wall particles, referred to as “whole glucan particles” or “WPGs” (See U.S. Pat. Nos. 5,032,401 and 5,607,677), may be modified to facilitate improved retention and/or delivery of payload molecules. Such improvements build on the art-recognized WGPs but feature trapping molecules and nanoparticles as well as pluralities of said trapping molecules and nanoparticles, formulated in specific forms to achieve the desired improved delivery properties. As used herein, a WGP is typically a whole glucan particle of >90% beta glucan purity.

Preparation of Glucan Particles (GPs)

Glucan particles (GPs), also referred to herein as yeast glucan particles (“YGPs”), are a purified hollow yeast cell ‘ghost’ containing a rich β-glucan sphere, generally 2-4 microns in diameter. GPs have been used for macrophage-targeted delivery of soluble payloads (DNA, siRNA, protein, and small molecules) encapsulated inside the hollow GPs via core polyplex and layer-by-layer (LbL) synthetic strategies.

In general, glucan particles were prepared from yeast cells by the extraction and purification of the alkali-insoluble glucan fraction from the yeast cell walls. The yeast cells were treated with an aqueous hydroxide solution without disrupting the yeast cell walls, which digests the protein and intracellular portion of the cell, leaving the glucan wall component devoid of significant protein contamination, and having substantially the unaltered cell wall structure of β(1-6) and β(1-3) linked glucans. The 1,3-β-glucan outer shell provides for receptor-mediated uptake by phagocytic cells, e.g., macrophages, expressing β-glucan receptors.

Certain glucan particles, were made as follows: yeast particles (S. cerevisiae), Biorigin MOS55 were suspended in 1 liter of 1M NaOH and heated to 85° C. The cell suspension was stirred vigorously for 1 hour at this temperature. The insoluble material containing the cell walls was recovered by centrifuging. This material was then suspended in 1M NaOH, heated, and stirred vigorously for 1 hour. The suspension was then allowed to cool to room temperature and the extraction can be continued for a further 16 hours. The insoluble residue was recovered by centrifugation. This material was finally extracted in water brought to pH 4.5 with HCl. The insoluble residue was recovered by centrifugation and washed three times with water, isopropanol, and acetone. The resulting slurry was placed in glass trays and dried under reduced pressure to produce a fine white powder.

Preparation of Glucan Lipid Particles (GLPs)

GLPs retain some of the yeast cellular lipid content which creates a more hydrophobic inner cavity ideal for loading of hydrophobic payloads. GLPs were prepared by modifying the method of preparation of GPs described above. For preparation of GLPs, washing with isopropanol and acetone was eliminated and instead the insoluble residue recovered by centrifugation was washed three times with water. The particles were dried by lyophilization or spray drying.

Commercial Yeast Particles (YPs)

Yeast particles (YPs) were purchased from Biorigin (Louisville, KY, USA) or LeSaffre (Milwaukee, WI). These YPs contained sufficient amounts of lipids to provide for a hydrophobic reservoir that attracts hydrophobic payloads to diffuse into the center of the particle accomplishing loading.

Whole Glucan Particles (WGPs)

A more detailed description of processes for preparing WPGs can be found in U.S. Pat. Nos. 4,810,646, 4,992,540, 5,028,703, 5,607,677, and 5,741,495 (incorporated herein by reference). For example, U.S. Pat. No. 5,028,703 discloses that yeast WGP particles can be produced from yeast strain R4 cells in fermentation culture. The WGPs were harvested by batch centrifugation at 8000 rpm for 20 minutes in a Sorval RC2-B centrifuge. The cells were washed twice in distilled water in order to prepare them for the extraction of the whole glucan. The first step involved resuspending the cell mass in 1 liter 4% w/v NaOH and heating to 100° C. The cell suspension was stirred vigorously for 1 hour at this temperature. The insoluble material containing the cell walls was recovered by centrifuging at 2000 rpm for 15 minutes. This material was then suspended in 2 liters, 3% w/v NaOH and heated to 75° C. The suspension was stirred vigorously for 3 hours at this temperature. The suspension was allowed to cool to room temperature and the extraction was continued for a further 16 hours. The insoluble residue was recovered by centrifugation at 2000 rpm for 15 minutes. This material was finally extracted in 2 liters, 3% w/v NaOH brought to pH 4.5 with HCl, at 75° C. for 1 hour. The insoluble residue was recovered by centrifugation and washed three times with 200 milliliters water, once with 200 milliliters dehydrated ethanol, and twice with 200 milliliters dehydrated ethyl ether. The resulting slurry was placed on petri plates and dried.

Varying degrees of purity of glucan particles can be achieved by modifying the extraction/purification process. As used herein, the terms YCWP, YGP, and GP describe a 2-4 micron intact hollow microsphere (or intact yeast cell wall ghost) purified from Baker's yeast using a series of alkaline, acid, and organic extraction steps as detailed supra. In general, these GPs are on the order of 80-85% pure on a w/w basis of beta glucan and, following the introduction of payload, trapping, or other components, become of a slightly lesser “purity.” In exemplary embodiments, GPs are <90% beta glucan purity.

Preparation of YCP Particles

Yeast cells (Rhodotorula sp.) derived from cultures obtained from the American Type Culture Collection (ATCC, Manassas, Va.) were aerobically grown to stationary phase in YPD at 30° C. Rhodotorula sp. cultures available from ATCC include Nos. 886, 917, 9336, 18101, 20254, 20837 and 28983. Cells were harvested by batch centrifugation at 2000 rpm for 10 minutes. The cells were then washed once in distilled water and then re-suspended in water brought to pH 4.5 with HCl, at 75° C. for 1 hour. The insoluble material containing the cell walls was recovered by centrifuging. This material was then suspended in 1 liter, 1M NaOH and heated to 90° C. for 1 hour. The suspension was allowed to cool to room temperature and the extraction continued for a further 16 hours. The insoluble residue was recovered by centrifugation and washed twice with water, isopropanol, and acetone. The resulting slurry was placed in glass trays and dried at room temperature to produce 2.7 g of a fine light brown powder.

In alternative embodiments, YGPs, e.g., activated YGPs, was grafted with chitosan on the surface, for example, to increase total surface chitosan. Chitosan can further be acetylated to form chitin (YGCP), in certain embodiments. Such particles can be seen to have equivalent properties when seen, in vivo, by the immune system of a subject or patient.

Preparation of YGMP Particles

S. cerevisiae (100 g Fleishman's Baker's yeast) were suspended in 1 liter 1M NaOH and heated to 55° C. The cell suspension was mixed for 1 hour at this temperature. The insoluble material containing the cell walls were recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with 1000 milliliters water, four times with 200 milliliters dehydrated isopropanol and twice with 200 milliliters acetone. The resulting slurry was placed in a glass tray and dried at room temperature to produce 12.4 g of a fine, slightly off-white, powder.

S. cerevisiae (75 g SAF-Mannan) were suspended in 1 liter water and adjusted to pH 12-12.5 with 1M NaOH and heated to 55° C. The cell suspension was mixed for 1 hour at this temperature. The insoluble material containing the cell walls were recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with water, dehydrated isopropanol, and acetone. The resulting slurry was placed in a glass tray and dried at room temperature to produce 15.6 g of a fine slightly off-white powder.

### Example 2: Loading of Cannabinoids in YPs/GLPs without Solvent

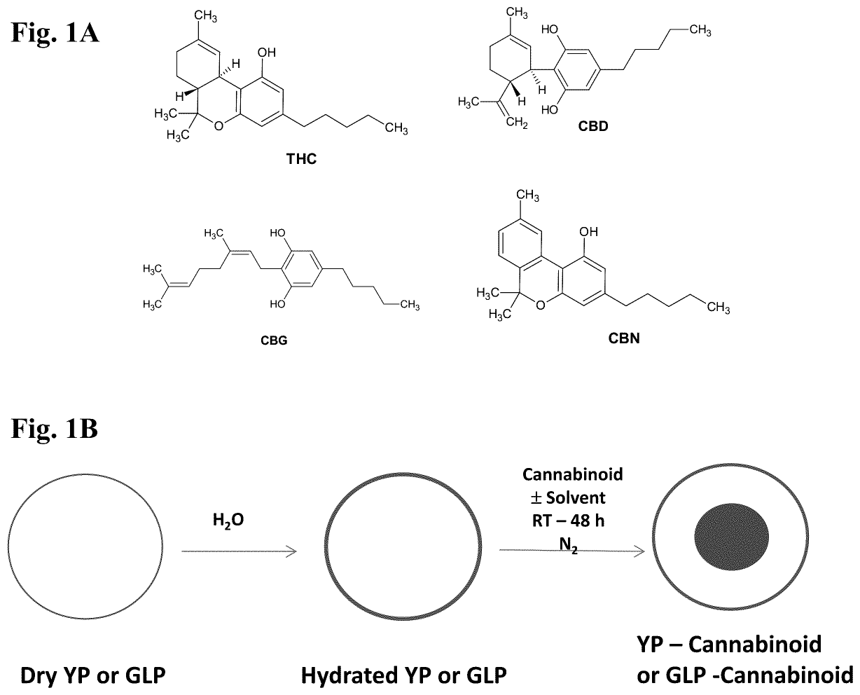

The porous cell wall structure of YPs/GLPs allows hydrophobic payloads like cannabinoids to be loaded from aqueous and some organic solutions with high payload loading capacity into the large hollow YP/GLP cavity. Cannabinoids like tetrahydrocannabinol (THC), cannabidiol (CBD), cannabigerol (CBG), and cannabinol (CBN) can be encapsulated in the hydrophobic cavity of GLPs by the passive diffusion of the payload through the YP shell into the interior hollow cavity. The structures of the cannabinoids used is shown in FIG. 1A and the schematic of loading cannabinoids in YPs/GLPs in the presence or absence of a solvent is shown in FIG. 1B.

Encapsulation Efficiency of Tetrahydrocannabiodiol (THC) in YPs/GLPs without a Solvent

Dry GLPs were mixed with water. Table 1 shows the amounts of water and GLPs used. The mixture was incubated overnight at 4° C. to obtain a homogenous hydrated GLP sample. THC was added to the hydrated GLP pellet at the THC:GLP weight ratios indicated in Table 1, the sample was purged with nitrogen and incubated 48+ hours at room temperature in the dark to allow for THC loading inside the hollow cavity of GLPs. The samples were lyophilized and then the dry GLP-THC pellet was mixed with 0.5 L water/g GLP and 2 L ethanol/g GLP, purged with nitrogen and incubated 24+ hours at room temperature in the dark to allow for loading of THC remaining on the surface of the particles after the first incubation step. The samples were lyophilized and the dry GLP THC pellet was purged with nitrogen and stored at 10° C. in the dark.

TABLE 1
Loading conditions and encapsulation efficiency of THC in GLPs.

| THC:GLP | | mL H₂O/g | Incubation | % THC |
|---|---|---|---|---|
| ratio | Particle | GLP | time at 23° C. | in GLPs |
| 1:1 | GLP | 4.5 | 2 days | 99.2 |
| 3:1 | GLP | 2.5 | 2 days | 99.4 |
| 4:1 | GLP | 1.5 | 2 days | 90.2 |
| 5:1 | GLP | 0.5 | 2 days | 81.0 |

Microscopy and Measurement of Average Particle Diameter of GLP-THCs

Samples of GLP-THC were stained with Nile red to qualitatively assess loading by the fluorescence microscopy of the encapsulated fluorescent THC-Nile red complex. Microscopy images of GLP control and GLP-THC samples were obtained at 1,000× magnification. An image of a microscope calibration slide ruler was used to set the scale in pixel/μm in ImageJ software. The photomicrographs of GLP samples were evaluated with the calibrated scale in ImageJ. The particle diameter along the major axis of the GLP ellipses was measured for 20 whole yeast cell particles per picture.

Quantification of THC Encapsulation in GLPs

GLP-THC samples (2 mg) were resuspended in 1 mL of water, centrifuged and the supernatant was collected to quantify free (emulsified) THC. The GLP THC pellet fraction was resuspended in 1 mL of 90% methanol-10% water and incubated at room temperature the dark for 24-48 hours to extract encapsulated THC. This THC extraction from GLP-THC was repeated three times to achieve mass balance. Other suitable solvents for THC extraction are 90% ethanol-10% water or 90% DMSO-10% water. Free and encapsulated THC was quantified by HPLC using a WATERS SYMMETRY® C18 column (3.5 μm, 4.6×150 mm) with acetonitrile:water 70:30 as mobile phase, flow rate at 1 mL/min, injection volume of 10 μL, and THC detection at 208 nm (THC retention time=10.2 minutes). HPLC method for quantification of cannabinoids is summarized in Table 2.

TABLE 2
HPLC assay conditions for quantitative analysis of cannabinoids.
HPLC method for quantification of cannabinoids

| | |
|---|---|
| Column | WATERS SYMMETRY ® C18, 3.5 μm, |
| | 4.6 × 150 mm |
| Mobile Phase | 70% acetonitrile - 30% water |
| Flow rate | 1 mL/min |
| Injection volume | 10 μL |
| Running time | 14 minutes |
| Detection | Absorbance @ 208 nm |
| Retention time | CBD: 5.0-5.2 minutes |
| | CBG: 4.9-5.0 minutes |
| | CBN: 8.6-8.7 minutes |
| | THC: 10.2 minutes |
| Linear range | CBD: 0-200 μg/mL |
| | CBG: 0-500 μg/mL |
| | CBN: 0-210 μg/mL |
| | THC: 0-250 μg/mL |

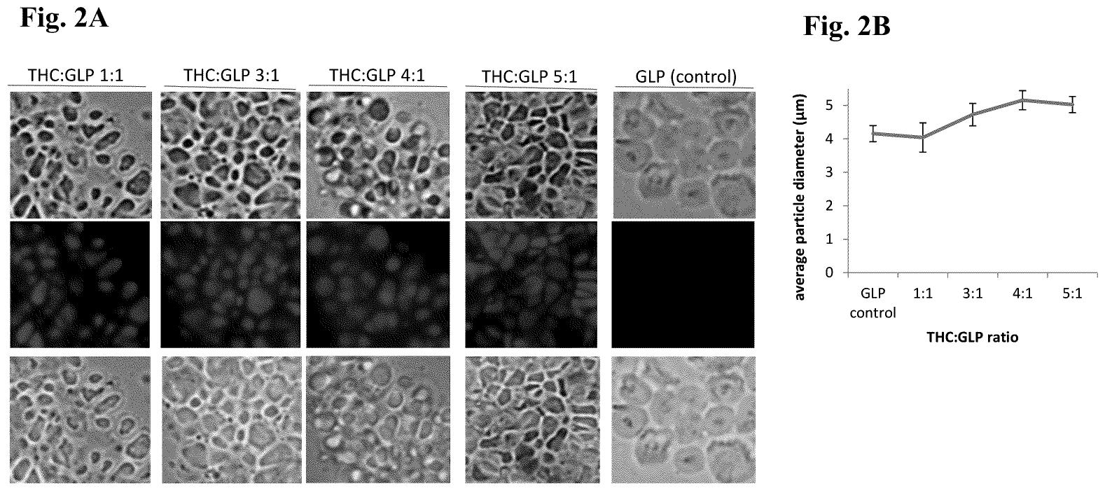

THC is an oil at room temperature and could be loaded into YPs/GLPs without a solvent. THC was encapsulated in GLPs with more than 90% efficiency to yield weight ratios of 4:1 THC:GLP (Table 1). Loading of THC led to swelling of GLPs as seen in light and fluorescent micrographs (FIG. 2A). Swelling of GLPs was corroborated by increase in particle size with increasing weight ratio of THC:GLP (FIG. 2B).

### Example 3: Loading of Cannabinoids in YPs/GLPs with a Solvent

Hydrophobic payloads (including cannabinoids) that are powders at room temperature can be loaded in YPs without solvent at temperatures above their melting point temperature provided the melting point is below 70° C. and the compound is thermally stable at the loading temperature. Other hydrophobic payloads to be dissolved in solvents prior to loading in YPs. Solvents may be used as “loading solvent” which is removed after payload is completely loaded in the YPs or solvents may be used as a “leave-in solvent” which remains inside with the payload. Non-limiting examples of solvents are ethanol, methanol, dimethylsulfoxide (DMSO), glycerin, fatty acids (e.g., octanoic, undecanoic, lauric acid), surfactants (NEOBEE®, TRITON™ X-100). Leave-in solvents may work as sequestering or releasing agents to control release of payloads from the YPs. Leave-in solvents reduce loading capacity of the cannabinoid payload.

Loading and Encapsulation Efficiency of THC in GLPs with Leave-In Solvent—Undecanoic Acid (UDA)

Dry GLPs were mixed with water. Amounts of water and GLPs used are indicated in Table 3. The mixture was incubated overnight at 4° C. to obtain a homogenous hydrated GLP sample. THC was added to the hydrated GLP pellet at the THC:GLP weight ratios indicated in Table 3 and the sample was purged with nitrogen and incubated for more than 48 hours at room temperature in the dark to allow for THC loading inside the hollow cavity of GLPs. The samples were lyophilized and then the dry GLP-THC pellet was mixed with 0.5 L water/g GLP and 2 L ethanol/g GLP, purged with nitrogen and incubated more than 24 hours at room temperature in the dark to allow for loading of THC remaining on the surface of the particles after the first incubation step. The samples were lyophilized and the dry GLP THC pellet was purged with nitrogen and stored at 10° C. in the dark.

TABLE 3
Loading conditions and encapsulation efficiency in
GLPs of THC and leave-in solvent undecanoic acid.

| THC:GLP | UDA:GLP | (THC + UDA):GLP | % THC in |
|---|---|---|---|
| ratio | ratio | ratio | GLPs |
| 1:1 | 0.5:1 | 1.5:1 | 98.6 |
| 1.5:1 | 0.5:1 | 2:1 | 96.5 |
| 2:1 | 0.5:1 | 2.5:1 | 86.9 |
| 1:1 | 1:1 | 2:1 | 99.6 |
| 1.5:1 | 1:1 | 2.5:1 | 94.2 |
| 2:1 | 1:1 | 3:1 | 46.7 |

Microscopy and measurement of average particle diameter was performed as described before. Free and encapsulated THC was quantified by HPLC as described before.

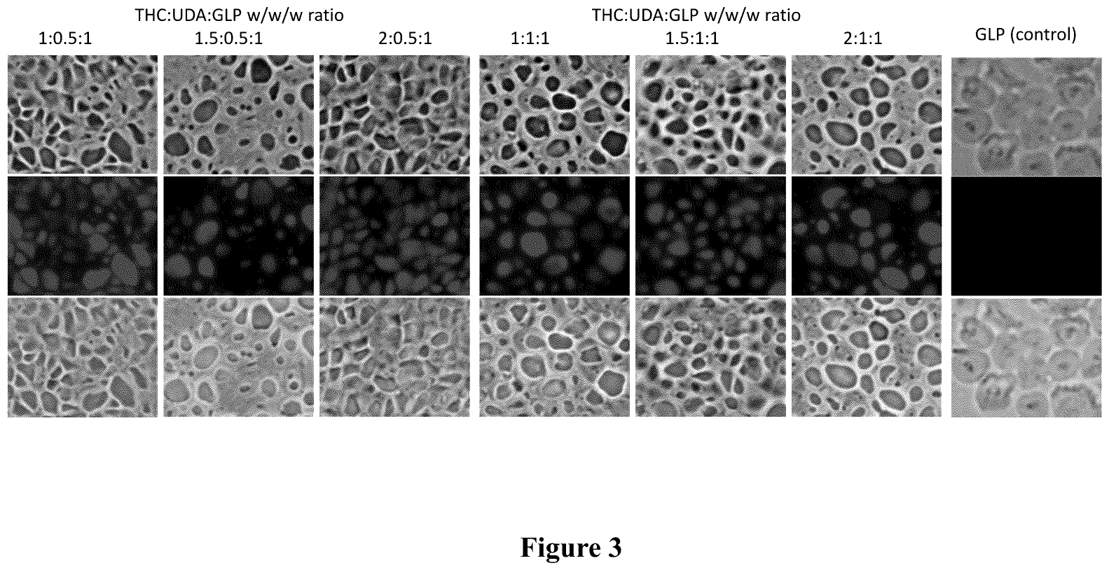

THC was co-encapsulated with UDA in GLPs with more than 85% efficiency to yield weight ratio of 2.5:1 (THC+UDA):GLP (Table 3). Light and fluorescent photomicrographs confirmed that THC and UDA were encapsulated within the GLPs (FIG. 3).

Loading and Encapsulation Efficiency of Cannabidiol in GLPs with Loading Solvent—Ethanol

Dry GLPs were mixed with water. The amount of water and GLP used are indicated in Table 4. Samples were incubated overnight at 4° C. to obtain a homogenous hydrated GLP sample. A solution of 0.5 g cannabidiol (CBD)/mL ethanol was added to the hydrated GLP pellet. The sample was mixed, purged with nitrogen and incubated 48 hours at room temperature in the dark. The GLP CBD sample was lyophilized and the CBD loading cycle was repeated until the target CBD:GLP weight ratio was achieved. The samples were lyophilized and stored as dry GLP CBD at 10° C. in the dark.

TABLE 4
Loading conditions and encapsulation efficiency in GLPs
of CBD using loading solvent ethanol.

| CBD: | mL | | g | mL | | | |
|---|---|---|---|---|---|---|---|
| GLP | H₂O/g | | CBD/mL | ethanol/ | Loading | Incubation | % CBD |
| ratio | GLP | Solvent | ethanol | g GLP | cycles | at 23° C. | in YP |
| 1:1 | 4.5 | Ethanol | 0.5 | 2 | 1 | 48 h | 99.8 |
| 3:1 | 2.5 | Ethanol | 0.5 | 3 | 2 | 48 h | 97.7 |
| 4:1 | 1.5 | Ethanol | 0.5 | 4 | 2 | 48 h | 96.1 |
| 5:1 | 0.5 | Ethanol | 0.5 | 2 | 3 | 48 h | 92.1 |

Microscopy and measurement of average particle diameter was performed as described before.

Quantification of CBD encapsulation: Dry GLP-CBD samples (10 mg GLP) were resuspended in 1 mL of 100% ethanol, centrifuged and the ethanol supernatant was collected to quantify unencapsulated CBD. The GLP-CBD pellet was resuspended in 1 mL of 80% ethanol −20% water and incubated at room temperature with constant mixing for 24 h. The sample was centrifuged and the supernatant collected to quantify extracted (GLP encapsulated) CBD. The GLP CBD incubation in 80% ethanol-20% water was repeated three times to completely extract CBD. The free and encapsulated CBD was quantified by HPLC using a WATERS SYMMETRY® C18 column (3.5 μm, 4.6×150 mm) with acetonitrile:water 70:30 as mobile phase, flow rate at 1 mL/min, injection volume of 10 μL, and THC detection at 208 nm (CBD retention time=5.0-5.2 minutes).

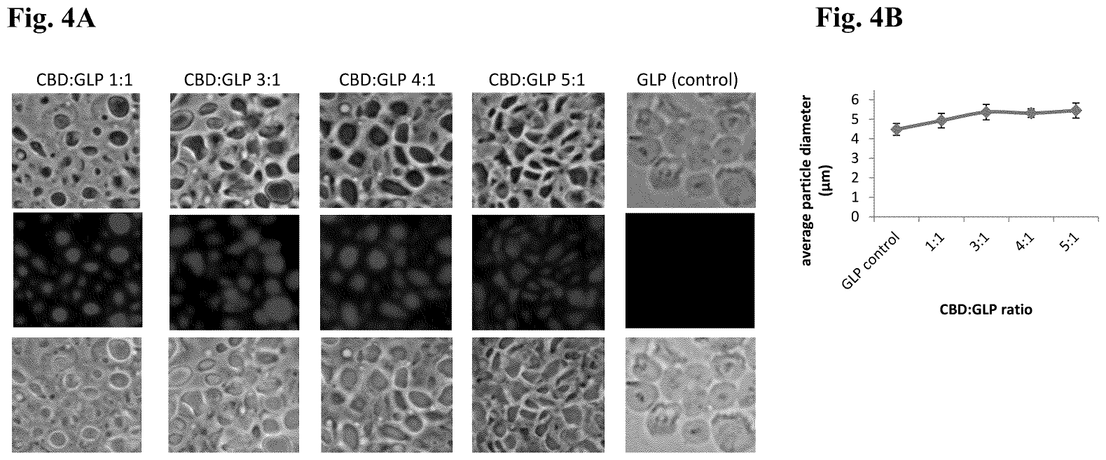

CBD was efficiently loaded in GLPs up to a ratio of 5:1 using ethanol as loading solvent (Table 4). Light and fluorescent photomicrographs confirmed that CBD and ethanol were encapsulated within the GLPs (FIG. 4A) and particle size measurements showed that hyper-loading of GLPs with CBD increased the average particle diameter (FIG. 4B).

Loading and Encapsulation Efficiency of Cannabidiol in GLPs with Leave-In Solvent—Octanoic Acid (OA)

Dry GLPs were mixed with water (2 mL water/g GLP) and incubated overnight at 4° C. to obtain a homogenous hydrated GLP sample. A solution of 0.5 g cannabidiol (CBD)/mL octanoic acid was added to the hydrated GLP pellet. The amount of cannabinoid, octanoic acid, and GLP used are indicated in Table 5. The sample was mixed, purged with nitrogen and incubated 48 hours at room temperature in the dark. The GLP-CBD-OA sample was lyophilized. The samples were lyophilized and stored as dry GLP CBD at 10° C. in the dark. This method can be applied to other cannabinoids that are solids at room temperature.

TABLE 5
Loading conditions and encapsulation efficiency in
GLPs of CBD using loading solvent octanoic acid.

| CBD:GLP | OA:GLP | (CBD + OA):GLP | Incubation | % CBD in |
|---|---|---|---|---|
| ratio | ratio | ratio | time 23° C. | GLP |
| 1:1 | 2:1 | 3:1 | 2 days | 100 |

Microscopy, measurement of average particle diameter, and quantification of CBD was performed as described before.

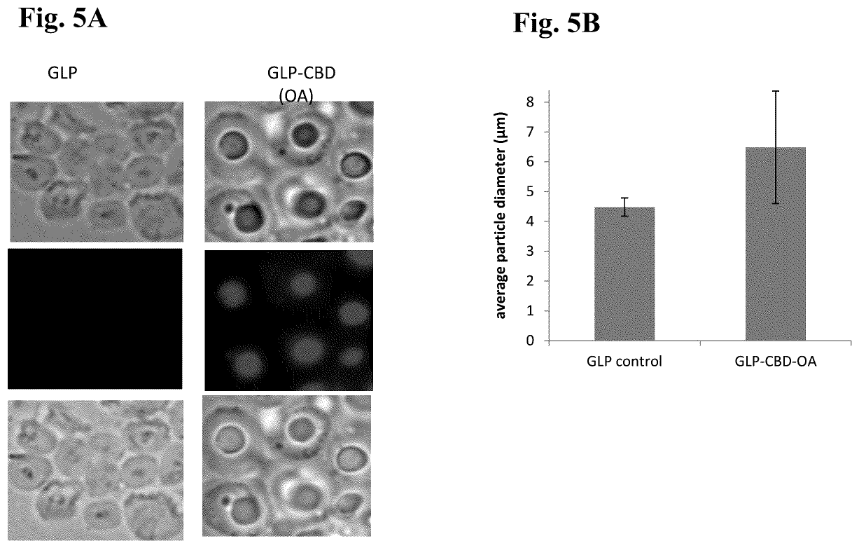

CBD was encapsulated at high (100%) efficiency in GLPs up to at a target weight ratio of 2:1:1 CBD:OA:GLP: using OA as leave-in solvent (Table 5). Light and fluorescent photomicrographs confirmed that CBD and OA were co-encapsulated within the GLPs (FIG. 5A) and particle size measurements showed that encapsulation of CBD and OA increased the average GLP diameter (FIG. 5B).

### Example 3: Co-Encapsulation of Cannabinol (CBN) and Melatonin in GLPs with Leave-In Solvent—Octanoic Acid (OA)/Glycofurol

Dry GLPs were mixed with water (1 mL water/g GLP) and incubated overnight at 4° C. to obtain a homogenous hydrated GLP sample. Loading solutions of cannabinol (CBN) in OA (150 mg CBN/mL) and melatonin in glycofurol (250 mg m/mL) were mixed at a ratio of 1:2 melatonin:CBN. The mixture was added to the hydrated GLP pellet. The ratios of CBN, melatonin, and GLP used are indicated in Table 6. The sample was mixed, purged with nitrogen and incubated 48 hours at room temperature in the dark. The GLP-CBN-OA-melatonin-glycofurol sample was lyophilized and stored as dry GLP CBD at 10° C. in the dark.

TABLE 6
Loading conditions and encapsulation efficiency in
GLPs of CBD using loading solvent octanoic acid.

| | | % | |
|---|---|---|---|
| | Melatonin:CBN:GLP | encapsulated | % encapsulated |
| Sample | weight ratio | Melatonin | CBN |
| GLP-Melatonin- | 0.25:0.5:1 | 81 ± 4 | 92 ± 1 |
| CBN | | | |

Microscopy and measurement of average particle diameter was performed as described before.

To quantify CBN and melatonin, dry GLP-CBN melatonin samples (10 mg GLP) were resuspended in 1 mL of 80% ethanol −20% water and incubated at room temperature with constant mixing for 24 h in the dark. The sample was centrifuged and the supernatant collected to quantify extracted CBN and melatonin. The extraction steps were repeated three times. An aliquot of the supernatants was diluted in 100% ethanol to quantify melatonin by measurement of melatonin fluorescence (excitation wavelength 285 nm, emission wavelength 340 nm) and a second aliquot was diluted in 100% methanol to quantify CBN by HPLC (CBN was analyzed by HPLC using the same method described for CBD in slide 11, CBN retention time=8.0-8.2 minutes).

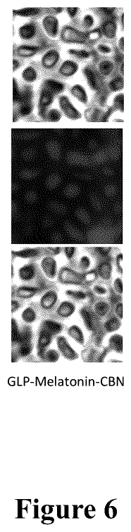

CBN and melatonin were co-encapsulated at high efficiency in GLPs with a leave-in solvent mixture of OA and glycofurol (Table 5). Light and fluorescent photomicrographs confirmed that CBN and melatonin were co-encapsulated within the GLPs (FIG. 6). This demonstrates that cannabinol and a non-cannabinoid payload (e.g., melatonin) can be co-loaded efficiently with a solvent mixture as leave-in solvent in GLPs.

### Example 4: Fast Release of Cannabinoids From GLPs

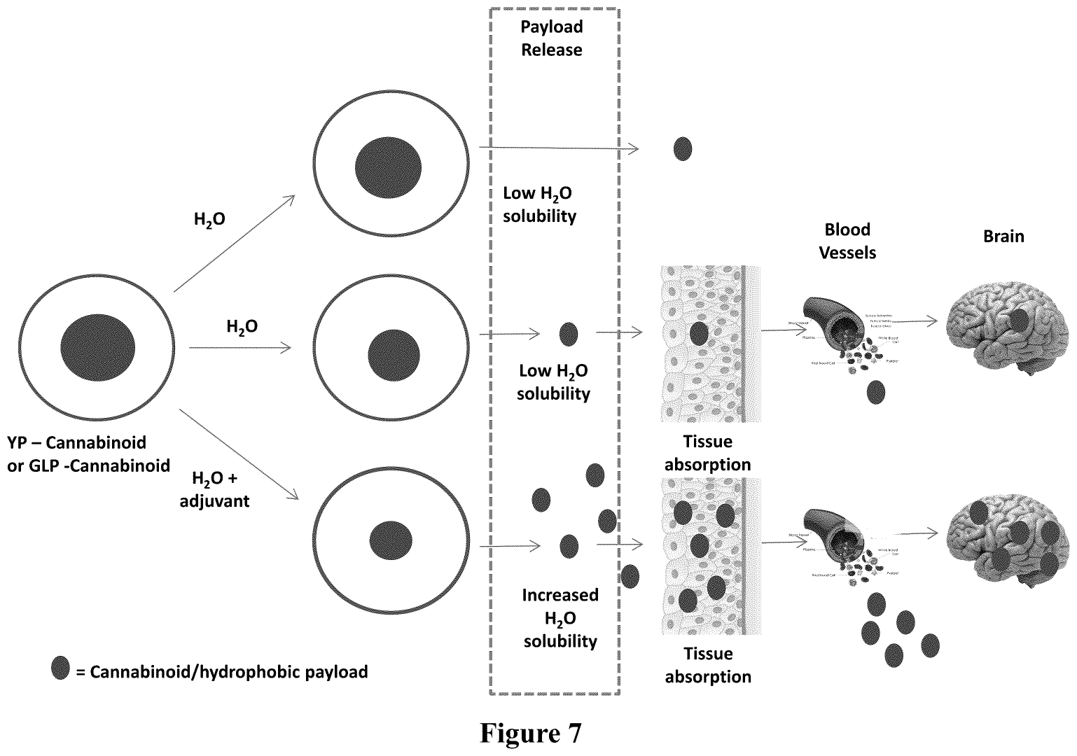

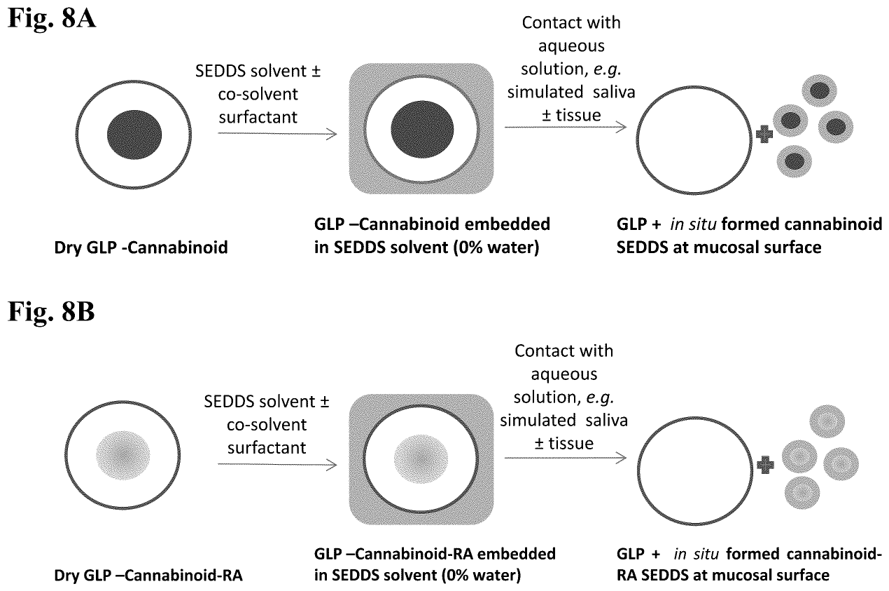

Cannabinoids encapsulated within YPs/GLPs can be released when diluted with water. FIG. 7 shows a schematic diagram of different strategies for cannabinoid release from GLPs. Cannabinoid release in vivo was controlled by addition of adjuvants to the payload carrying YPs/GLPs. Adjuvants can be solvents, surfactants, or a combination of the two. Suitable solvents can include organic solvents such as alcohols, fatty acids or terpenes. Surfactants may be lecithin, TRITON™ X100, BRIJ® surfactants or PLURONIC® surfactants. Solvents and surfactants can allow controlled payload release by modulating the water solubility of the payload. Combination of certain solvents and surfactants can create a self-emulsifying drug delivery system (SEDDS) to release cannabinoids or other lipophilic/hydrophobic drug compounds from YPs/GLPs. SEDDS contains an isotropic mixture of oils, surfactants, solvents and co-solvents. SEDDS improves the bioavailability and absorption of highly lipophilic drug compounds such as cannabinoids. Payload carrying YPs/GLPs delivered in SEDDS can release payload within seconds upon contact with water/aqueous solution. Payload release can be further enhanced if encapsulated payload contains a release agent such as a fatty acid. FIG. 8 shows the schematic of payload release using a SEDDS. Examples of SEDDS solvents include glycerin and 1,3-propanediol. Examples of SEDDS co-solvents include fatty acids, medium chain triglycerides (MCT). Examples of SEDDS surfactants include lecithin. Examples of release agents include fatty acids such as undecanoic acid and octanoic acid.

Release of Cannabinoids from GLPs Embedded in Adjuvant

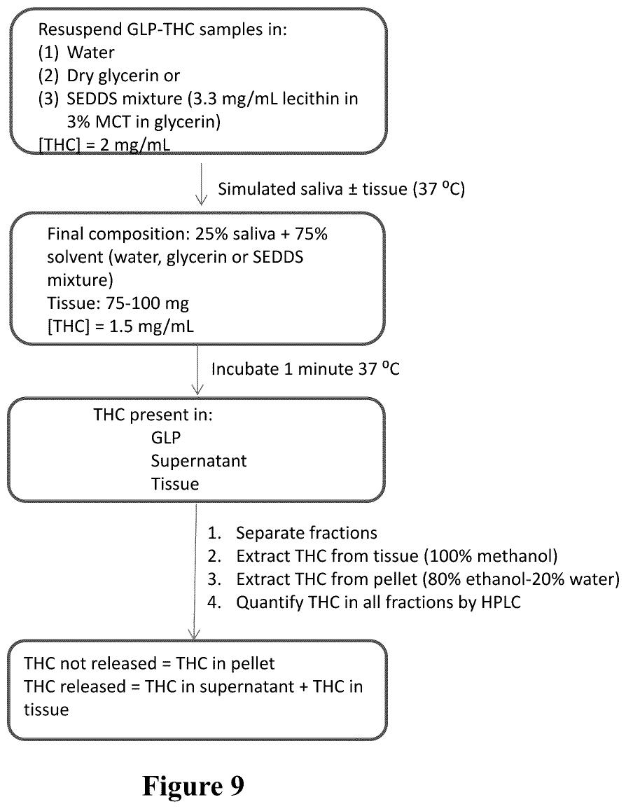

FIG. 9 shows the flow chart of steps to release and quantify cannabinoids from GLPs embedded in different adjuvants/solvents. Composition of simulated saliva was 0.72 g/L potassium chloride, 0.22 g/L calcium chloride dihydrate, 0.68 g/L potassium phosphate monobasic, 0.866 g/L sodium phosphate dibasic, 1.5 g/L potassium bicarbonate, 0.06 g/L potassium thiocyanate, and 0.03 g/L citric acid. Samples of dry GLP-THC (4 mg) were resuspended in 1 mL of (A) water, (B) dry glycerin or (C) SEDDS mixture (3.3 mg/mL lecithin in 3% MCT in glycerin). The GLP THC suspensions were diluted with simulated saliva (25% simulated saliva+75% GLP THC in solvent mixture) with and without the addition of a sample of 75-100 mg fat tissue. The samples were incubated for 1 minute at 37° C., and then the fractions were separated to quantify THC in the GLP pellet (THC not released), THC in supernatant (released) and THC in the tissue (released from GLP and absorbed by the tissue). The THC absorbed in the tissue was extracted in 100% methanol and the THC remaining in GLPs was extracted in 80% ethanol-20% water. The THC in all fractions was quantified by HPLC.

Fast Release of THC from GLPs Loaded with THC:GLP Weight Ratio of 1:1

GLPs were loaded with a THC:GLP weight ratio of 1:1 and embedded in different solvents. THC release was achieved and quantified as described before (FIG. 9).

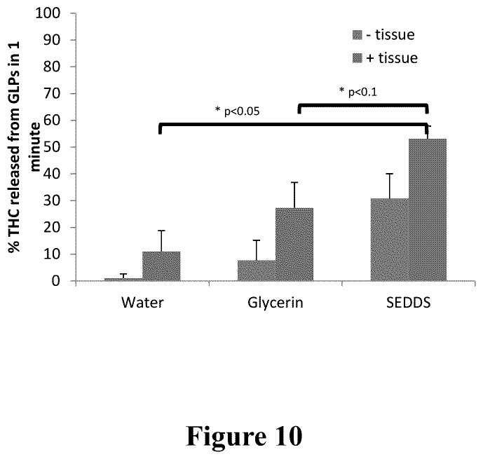

FIG. 10 shows the amount of THC (average of six experiments) released from GLPs upon contact with simulated saliva. Up to 30% THC was released in 1 minute when GLPs embedded in glycerin were incubated with simulated saliva containing tissue. GLPs embedded in SEDDS mixture (lecithin/MCT/glycerin) also released up to 30% THC when incubated with simulated saliva without tissue. However, addition of tissue to the simulated saliva increased THC release up to 53% THC from GLPs were embedded in SEDDS mixture.

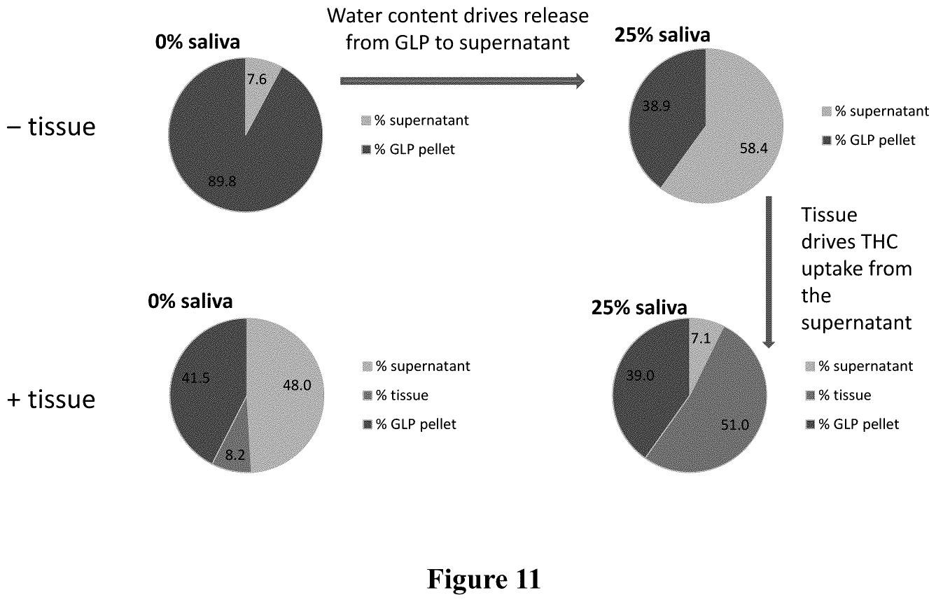

GLP were embedded in SEDDS mixtures. THC in various fractions was quantified in the presence and absence of tissue. Simulated saliva was then added and THC in various fractions was quantified after contact with saliva. FIG. 11 depicts the percentage of THC in each fraction. Results suggest that the water content of simulated saliva/tissue drives release of THC from GLPs via in situ SEDDS formation. Tissue drives uptake of free THC from the supernatant resulting in sequestration of THC in the tissue.

These results suggest that YPs/GLPs can be effective delivery vehicles for cannabinoids that can bring about fast release cannabinoids (in less than 1 minute) upon contact with bodily fluids/tissues.

Fast Release of THC from GLPs Loaded with THC and a Release Agent

GLPs were loaded with a THC and undecanoic acid as a release agent (RA). Loaded GLPs were embedded in adjuvants (water, glycerin or SEDDS mixture). THC release was achieved and quantified as described before (FIG. 9).

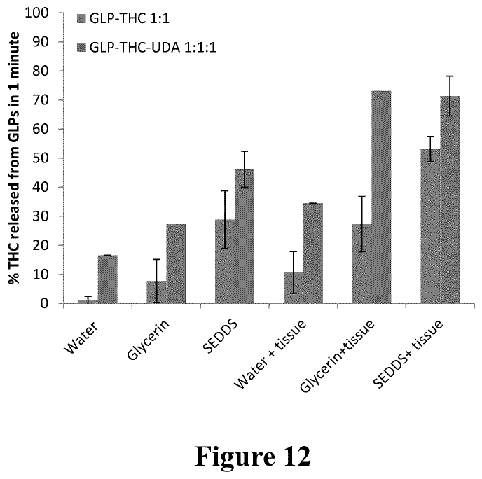

FIG. 12 shows the amount of THC released from GLPs upon contact with simulated saliva. Co-loading THC with UDA as a RA improves fast THC release from particles in GLP-THC-UDA samples embedded in glycerin or SEDDS mixture. Up to 30% THC release is possible in 1 minute in glycerin+tissue or SEDDS solvent (lecithin/MCT/glycerin) without tissue, and up to 53% THC release in 1 minute with SEDDS solvent and tissue. THC was released from GLPs by water activated in situ THC SEDDS formation

Fast Release of Cannabidiol (CBD), Cannabinol (CBN) and Cannabigerol (CBG) a Co-Loaded with the RA Octanoic Acid (OA)

GLPs were loaded with a CBD, CBN or CBG and octanoic acid as a RA. Loaded GLPs were embedded in adjuvant water or SEDDS mixture. Cannabinoid release was achieved and quantified as described before for THC (FIG. 9).

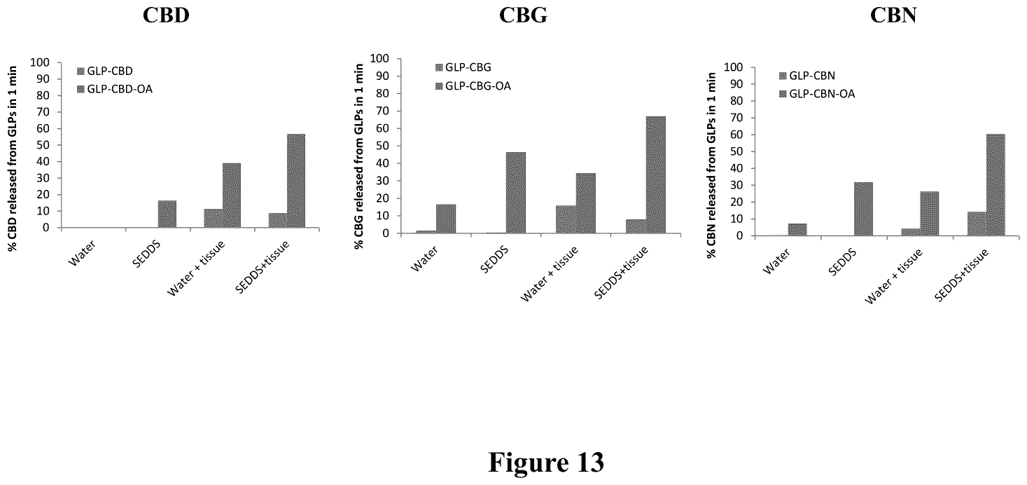

CBD, CBN and CBG are all powders at room temperature and do not release efficiently from GLP in 1 minute. Co-loading cannabinoids with RA kept cannabinoid soluble inside GLPs and significantly improved release from GLPs in 1 minute upon contact with simulated saliva with or without tissue. FIG. 13 shows the amounts of cannabinoids released from GLPs upon contact with simulated saliva with or without tissue. Co-loading cannabinoids with OA as a RA improves fast THC release from particles in GLP-Cannabinoid-OA samples embedded in water or SEDDS mixture. Cannabinoids were released from GLPs by water activated in situ cannabinoid-SEDDS formation.

### Example 5: Sustained Cannabinoid Release from GLPs

As seen in Example 4, GLPs embedded in adjuvant SEDDS mixture effectively released cannabinoids within 1 minute upon contact with water. Up to 50-60% of cannabinoids were released in the absence of a release agent and the release increased to 70-80% when a RA was co-loaded into the GLPs along with the cannabinoids. The amount of cannabinoid release plateaued at 1 minute. A 10-20% increase was measured after 24 h.

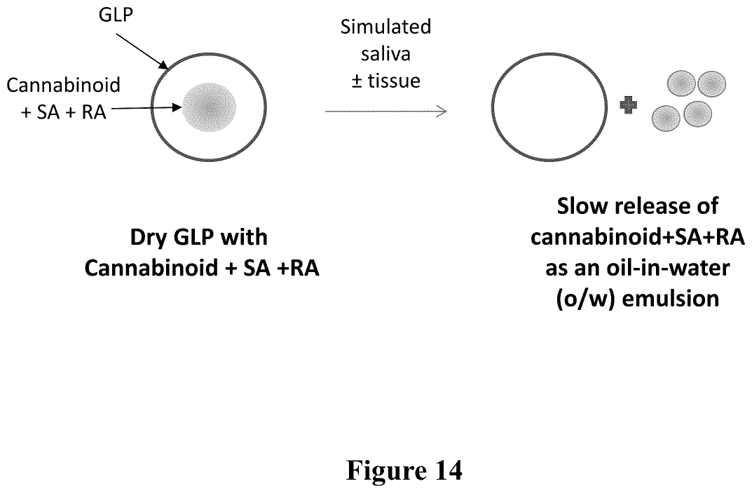

To ensure that YP/GLPs slowly release cannabinoids over a period of 1 to 24 hours after YP/GLP delivery, a sequestering agent (SA) was added to the payload with or without a RA. Examples of SAs are fatty acids, fatty acid ester mixtures (CAPMUL®, NEOBEE®), terpenes. RAs compatible with SAs include fatty acids, fatty acid ester mixtures (CAPMUL®, NEOBEE®), and surfactants (TRITON™ X100, BRIJ® surfactants, PLURONIC® surfactants). Payloads co-loaded with SAs (±RA) into GLPs were slowly released from the GLPs as an oil-in-water emulsion to give sustained release over a period of several hours after contact with an aqueous solution such as simulated saliva (FIG. 14).

Sustained CBD Release from GLP with Octanoic Acid as a SA

GLPs were loaded at a weight ratio of CBD:OA:GLP of 2:1:1. Dry GLP-CBD (5 mg) were resuspended in 1 mL of simulated saliva, a 75-100 mg piece of simulated tissue was added to the sample and incubated at 37° C. with constant mixing. Aliquots (200 μL) were collected at 1, 3, 6 and 24 h and the samples were centrifuged to separate supernatant (released CBD fraction) and GLP pellet. Released CBD was quantified in the supernatants by HPLC as described before.

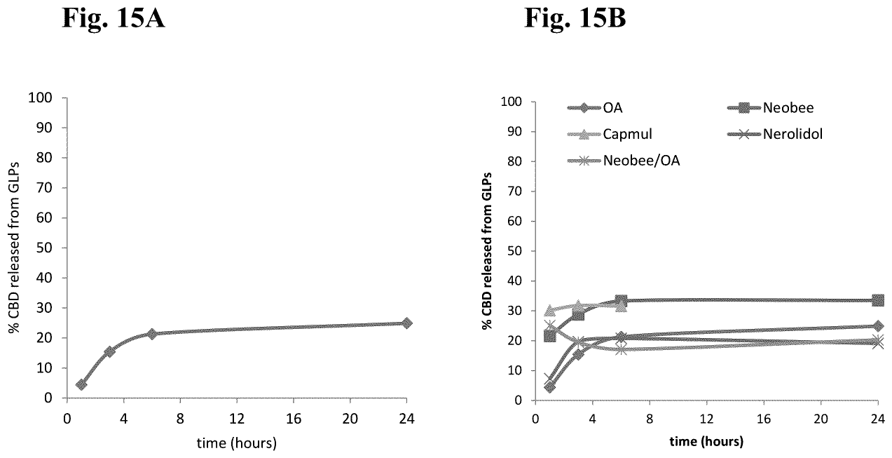

After contact with simulated saliva (without simulated tissue), CBD release plateaued after 6 hours and a maximum of 25% CBD was released after 24 h incubation (FIG. 15A). CBD release was improved with use of different sequestering agents.

CBD was co-loaded into GLPs with other SAs (NEOBEE® 1053, CAPMUL®, Nerolidol and a mixture of NEOBEE® 1053 and OA) using weight ratios indicated in Table 7.

TABLE 7
Weight ratios of CBD, SAs and GLPs.
Sample composition w/w ratio

GLP-CBD-OA 1:1:2
GLP-CBD-NEOBEE ® 1053 1:1:2
GLP-CBD-CAPMUL ® 1:1:2
GLP-CBD-Nerolidol 1:1:1
GLP-CBD-NEOBEE ® -OA 1:1:1:1

NEOBEE® 1053, CAPMUL® and the OA/NEOBEE® 1053 mix improved CBD burst release (1 h). NEOBEE® 1053 yielded −12% additional CBD release after 24 hours (FIG. 15B). Overall, none of the SAs tested resulted in a significant increase of CBD release after 1 h. Inclusion of a release agent (RA) improved sustained release by stabilization of the oil-in-water emulsion (payload-SA micelles).

Selection of RAs for Stabilization of Payload-SA Micelles

Compounds with a hydrophilic-lipophilic balance (HLB) values of 8-16 are conducive to making oil-in-water (o/w) emulsions. These compounds make good RAs by stabilizing payload-SA micelles. Table 8 shows the properties of various surfactants evaluated for the potential to be a good RAs to enhance sustained release of CBD.

TABLE 8
Properties of surfactants tested as RAs.

| | | Maximum | Stable CBD- | |
|---|---|---|---|---|
| | | solubility | OA-RA o/w | Micelle size |
| Surfactant | HLB* | in OA | emulsion ** | (nm) |
| OA only (control) | — | | No | 379 |
| PLURONIC ® 31R1 | 2.0-7.0 | 30% w/w | No | n/a |
| Lecithin | 8 | 15% w/w | No | n/a |
| PLURONIC ® 17R4 | 7-12 | 30% w/w | Yes | 258 |
| IGEPAL CO-520 | 10 | 30% w/w | Yes | 108 |
| TRITON ™ X-100 | 13.5 | 20% w/w | Yes | 83 (85%) |
| | | | | and |
| | | | | 340 (15%) |
| TWEEN 80 | 15 | 25% w/w | No | n/a |
| Brij 58 | 16 | 10% w/w | Yes | 220 |
| TWEEN 20 | 16.7 | 20% w/w | No | n/a |
| Brij 35 | 17 | 7.5% w/w | Yes | 357 |
| Saponin | N/A | Not | N/A | n/a |
| | | soluble | | |

*HLB: Hydrophilic-lipophilic balance.
** CBD-OA-RA emulsions were prepared at 1 mg/mL in water by sonication and stored at room temperature for 30 minutes. Samples were evaluated for formation of precipitate and/or phase partitioning that indicates an unstable emulsion.

PLURONIC 17R4, IGEPAL CO-520, TRITON™ X-100, Brij58, and Brij35 form stable micelles. TRITON™ X-100 generates micelles of smaller particle size (85% of micelles in CBD-OA-TRITON™ X100 samples have an average particle size of 83 nm). Thus, these compounds have the potential to serve as good RAs to increase sustained release of CBD.

The effect of addition of RAs to CBD with OA as SA was tested. Different RAs were added to CBD-OA preparation in weight ratios shown in Table 9.

TABLE 9
Weight ratios of release agents, CBD, and OA loaded in GLPs.
Sample composition w/w ratio

GLP-CBD-OA 1:1:2
GLP-CBD-OA-PLURONIC 17R4 1:1:1.5:0.5
GLP-CBD-OA-TRITON ™ X100 1:1:2:0.5
GLP-CBD-OA-Brij58 1:1:2:0.15
GLP-CBD-OA-Brij35 1:1:2:0.15

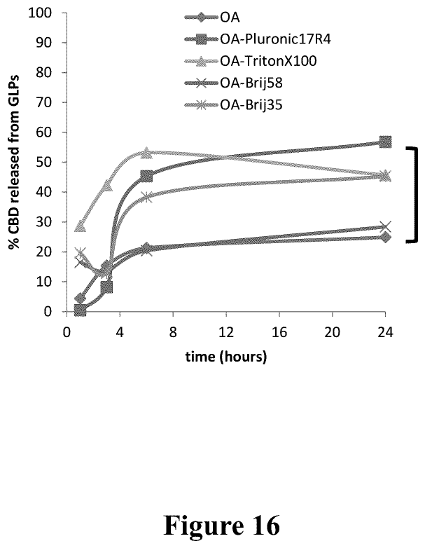

GLPs were loaded with CBD, OA and various RAs. Release assays were performed by the procedure described as before. RA PLURONIC® 17R4 inhibits CBD release at 1 and 3 hours and there is a significant increase of CBD release from 3 to 6 hours (FIG. 16). TRITON™ X-100 shows improvement of sustained release from 1 to 6 hours and a 1.7-2.0 fold increase of CBD release compared to GLP-CBD-OA control sample.

Stabilization of CBD-SA Micelles with TRITON™ X-100

The ability of TRITON™ X-100 to stabilize payload (CBD) micelles formed with other SAs besides OA was also tested. Table 10 shows the amount of TRITON™ X-100 used and the micelle size and stability achieved with three different SAs.

TABLE 10
Size and stability of micelles formed with different
SAs using TRITON ™ X-100 as the RA

| | | | Stable CBD | CBD-SA-RA |
|---|---|---|---|---|
| | | % w/w | o/w | micelle |
| SA | RA | surfactant | emulsion* | size (nm) |
| Octanoic acid | — | — | No | 379 |
| Octanoic acid | TRITON ™ | 20 | Yes | 83 (85%) and |
| | X100 | | | 340 (15%) |
| NEOBEE ® | — | — | Yes | 188 |
| 1053 | | | | |
| NEOBEE ® | TRITON ™ | 20 | Yes | 76.2 |
| 1053 | X100 | | | |
| Nerolidol | — | — | No | 1260 |
| Nerolidol | TRITON ™ | 20 | Yes | 661 |
| | X100 | | | |

*CBD-SA-RA emulsions were prepared at 1 mg/mL in water by sonication and stored at room temperature for 30 minutes. Samples were evaluated for formation of precipitate and/or phase partitioning that indicates an unstable emulsion.

Addition of TRITON™ X100 reduced the size of micelles formed with every SAs tested and increased micelle stability (Table 10). The smallest micelle size (average particle size <100 nm) was obtained when CBD was dissolved in octanoic acid or NEOBEE® 1053 as SAs and TRITON™ X100 as the RA.

Sustained CBD Release from GLP with Different SAs and TRITON™ X-100 as the RA

GLPs were loaded with CBD and SA at weight ratios shown in Table 11. Loaded GLPs (5 mg) were resuspended in 1 mL of simulated saliva, a 75-100 mg piece of simulated tissue was added to the sample and incubated at 37° C. with constant mixing. Aliquots (200 μL) were collected at 1, 3, 6 and 24 h and the samples were centrifuged to separate supernatant (released CBD fraction) and GLP pellet. Released CBD was quantified in the supernatants by HPLC as described before.

TABLE 11
Weight ratios of CBD, SAs, RAs and GLPs.
Sample composition w/w ratio

GLP-CBD-OA 1:1:2
GLP-CBD-OA-TRITON ™ X-100 1:1:2:0.5
GLP-CBD-NEOBEE ® 1053 1:1:2
GLP-CBD-NEOBEE ® 1053-TRITON ™ X100 1:1:1:1
GLP-CBD-Nerodiol 1:1:1
GLP-CBD-Nerolidol-TRITON ™ X-100 1:1:1

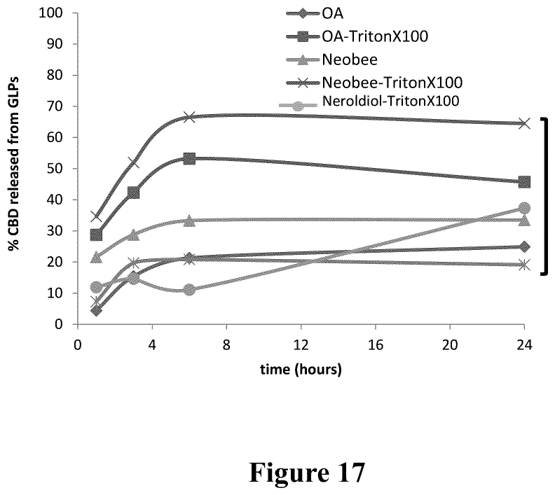

When a combination of NEOBEE® as SA and TRITON™ X-100 as SA was used, the release of CBD from GLPs was enhanced leading to release of up to 65% of total CBD in GLPs being released (FIG. 17). CBD release reached a maximum at around 6 hours. There was no significant increase in release at the 24 hour time point, likely due to the remaining CBD trapped as large CBD-NEOBEE® 1053-TRITON™ X100 micelles. With Nerolidol as the SA, TRITON™ X-100 did not lead to CBD release in the first 6 hours after contact with saliva and tissue but produced a steady release from 6 to 24 hours to yield a total release of 37% at 24 hours (FIG. 17).

Selection of RAs for Stabilization of THC-UDA Micelles

Various RAs were tested as described before for their ability to stabilize micelles formed when GLPs encapsulate THC as the payload and UDA as the SA. Table 11 shows the properties of various surfactants evaluated for the potential to be a good RAs to enhance sustained release of THC.

TABLE 12
Properties of surfactants tested as RAs
for stabilization of THC-UDA micelles.

| | | | Physical State |
|---|---|---|---|
| | | | of THC-UDA- |
| | | Maximum | RA mixture at |
| | | solubility in | room |
| Surfactant | HLB* | UDA | temperature |
| UDA only (control) | — | — | Liquid |
| PLURONIC ® 31R1 | 2.0-7.0 | 43.2% w/w | Solid |
| PLURONIC ® 17R4 | 7-12 | 37.5% w/w | Solid |
| TRITONTM X-100 | 13.5 | 37.5% w/w | Solid |
| Brij 58 | 16 | 33.3% w/w | Yes |
| Brij 35 | 17 | 33.3% w/w | Liquid |

*HLB: Hydrophilic-lipophilic balance.

The effect of addition of RAs to THC with UDA as SA was tested. Different RAs were loaded in GLPs along with THC-UDA at weight ratios shown in Table 13. Release assays were performed as described as before.

TABLE 13
Weight ratios of different RAs with CBD and UDA
used for loading GLPs.
Sample composition w/w ratio

GLP-THC-UDA 1:1:1
GLP-THC-UDA- PLURONIC ® 17R4 1:1:0.6:0.4
GLP-THC-UDA-TRITON ™ X100 1:1:0.6:0.4
GLP-THC-UDA-Brij35 1:1:0.7:0.3

Sustained release of THC was improved by addition of a RA to GLP-THC-UDA samples. Brij35 improves sustained release from 1 h to 24 h. THC-UDA-Brij35 is a stable oil at room temperature and likely quickly form micelles upon resuspending GLP sample in saliva. PLURONIC® 17R4 and TRITON™ X100 did not improve sustained release from 1 to 6 h, but yielded high THC release at the 24 hour time point. The delay in THC release can be explained by the fact that THC-UDA mixtures with PLURONIC® 17R4 and TRITON™ X100 are less stable (mixture of oil and solid UDA) and release requires two steps: (1) solubilization of THC-UDA-RA mixture at 37° C. and (2) emulsification in simulated saliva inside GLPs.

Sustained Simultaneous Release of CBD and Melatonin Co-Loaded in GLPs

CBD and melatonin were co-loaded in GLPs with a solvent mixture of octanoic acid and glycofurol as described before and loaded GLPs were embedded in dry glycerin. Release assays were performed as described before. Briefly, GLP-glycerin formulation was incubated with simulated saliva with or without tissues at 37° C. CBD and melatonin released from GLPs at different time points over four hours was quantified by HPLC.

Up to 60% of each of the two payloads (CBD and melatonin) were released from GLPs after four hours of incubation with simulated saliva and tissue. These results corroborated that two distinct payloads were effectively co-encapsulated in GLPs and effectively released upon contact with water.

### Example 6: Storage Stability of Payload in GLPs

The mechanism of release of payloads encapsulated in GLPs/YPs is driven by contact with water. Water swells the GLP/YP shell increasing the pore size allowing diffusion of the payload out of the particle. Thus, payload stability can be compromised if the loaded GLPs/YPs come in contact with water or humidity during storage. To enhance long-term storage stability of GLP encapsulated payloads, drying agents or desiccants were used to induce or sustain a state of dryness in the YP preparation. Chemicals like sodium carbonate, calcium sulfate, and calcium chloride act as good drying agents and are FDA approved food additives.

Room Temperature Encapsulation Stability of THC in GLPs

Glycerin was dried with molecular sieves. Dry samples of GLP-THC (4 mg) were resuspended in 400 μL of dry glycerin or dry glycerin containing 10% w/w drying agent (drying agents: calcium sulfate, sodium carbonate). The samples were transferred to glass vials, purged with nitrogen and incubated at room temperature in the dark. Samples were collected after 1-, 2- and 4-weeks incubation. The amount of THC released from the particles into the glycerin supernatant was quantified by HPLC and the THC that remained encapsulated in GLPs was extracted in 80% ethanol-20% water and quantified by HPLC.

Combination of calcium sulfate and glycerin improved THC storage encapsulation stability in GLPs at room temperature up to 1 month. Data in Table 14 shows that when calcium sulfate was present as the drying agent, THC was chemically stable in all GLP-THC samples stored in glycerin or dry glycerin at room temperature (only 1.1±0.7% THC was decomposed to CBN after one week, 1.8±0.9% after two weeks, 2.4±1.4% after four weeks).

TABLE 14
Effect of drying agents on Room temperature
encapsulation stability of THC in GLPs.

| | | | % encapsulated THC in GLP-THC | | |
|---|---|---|---|---|---|
| | | | after incubation in 100% glycerin | | |
| | | % | t = 1 | t = 2 | t = 4 |
| | Drying | encapsulated | week | weeks | weeks |
| Solvent | agent | THC (t = 0) | 23° C. | 23° C. | 23° C. |
| Glycerin | — | 98.5% | 90.8 | 73.0 | 73.4 |
| Dry | — | 98.5% | 92.5 | 83.2 | 84.3 |
| glycerin | | | | | |
| Glycerin | Calcium | 98.5% | 97.5 | 91.6 | 92.2 |
| | sulfate | | | | |
| Dry | Calcium | 98.5% | 97.5 | 98.5 | 91.9 |
| glycerin | sulfate | | | | |
| Glycerin | Sodium | 98.5% | 97.8 | 64.5 | — |
| | carbonate | | | | |
| Dry | Sodium | 98.5% | 91.2 | 82.6 | — |
| glycerin | carbonate | | | | |

The result indicates that cannabinoids loaded in GLPs remained stable at room temperature for up to one month.

### Example 7: Loading of Oleamide in YPs/GLPs with a Solvent

To test loading efficiency oleamide in GLPs, oleamide was dissolved in an organic solvent prior to loading in YPs. Organic solvents such as ethanol, methanol, dimethylsulfoxide (DMSO), glycerin, fatty acids (e.g., octanoic, undecanoic, lauric acid), and surfactants (NEOBEE®, TRITON™ X-100) were used. The solubility of oleamide in the organic solvent determined the maximum loading capacity. The organic solvent was used either as “loading solvent” or as a “leave-in solvent”. When the organic solvent was used only as a loading solvent, it was subsequently removed. This loading cycle was repeated two or more times to achieve high oleamide:GLP weight ratio. When used as a “leave-in solvent,” a non-toxic organic solvent was chosen. Loading cycles could not be repeated when a leave-in solvent was used. The leave-in solvent can also serve as a sequestering or releasing agent, thereby controlling the release of oleamide from the GLPs.

Loading and Encapsulation Efficiency of Oleamide in GLPs with Leave-In Solvents—Undecanoic Acid (UDA)

Dry GLPs were mixed with water. The amounts of water and GLPs that were used are indicated in Table 1. The mixture was incubated overnight at 4° C. to obtain a homogenous hydrated GLP sample. Oleamide was added to the hydrated GLP pellet at the Oleamide:GLP weight ratios of 0.25:1, 0.5:1, or 1:1 as indicated in Table 15. The samples were purged with nitrogen and incubated in the dark for more than 48 hours at the temperature indicated in Table 15 to allow loading of oleamide into GLPs. The samples were lyophilized and then the dry GLP-oleamide pellets were mixed with 0.5 L water/g GLP and 2 L ethanol/g GLP, purged with nitrogen and incubated in the dark for more than 24 hours at said temperature to allow loading of oleamide remaining on the surface of the particles after the first incubation step. The samples were lyophilized and the dry GLP oleamide pellets were purged with nitrogen and stored at 10° C. in the dark.

Microscopy and measurement of average particle diameters were performed as described before. Free and encapsulated oleamide were quantified by HPLC as described above.

Oleamide was co-encapsulated with leave-in solvent in GLPs with 100% or near 100% encapsulation efficiency when undecanoic acid, myristic acid, palmitic acid, lauric acid, octanoic acid or oleic acid were used as leave-in-solvent (Table 15). Geraniol and eugenol were not as effective as other leave-in solvents, providing 47% and 70% encapsulation efficiency, respectively (Table 15).

TABLE 15
Loading conditions and encapsulation efficiency
in GLPs of oleamide using leave-in solvents.

| | oleamide | Loading | % oleamide encapsulation | | |
|---|---|---|---|---|---|
| Solvent | solubility | temper- | efficiency | | |
| (leave-in | (mg/mL) in | ature | Oleamide:GLP weight ratio | | |
| solvents) | solvent | (° C.) | 0.25:1 | 0.5:1 | 1:1 |
| Chloroform | 50 | 23 | 95 | — | — |
| Geraniol | 100 | 23 | — | 49 | — |
| Eugenol | 100 | 23 | — | 70 | — |
| Octanoic acid | 400 | 23 | — | 99.8 | 90 |
| Undecanoic acid | 400 | 37 | — | 100 | 90 |
| Lauric acid | 400 | 45 | — | 99.9 | 98 |
| Myristic acid | 400 | 60 | — | 100 | 97 |
| Palmitic acid | 400 | 65 | — | 100 | 95 |
| Oleic acid | 250 | 23 | — | 100 | 89 |

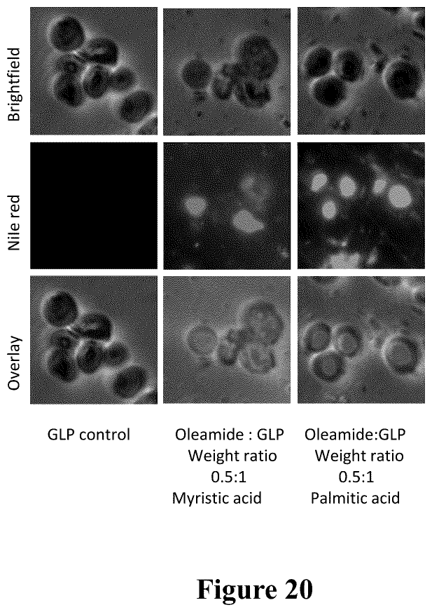

FIG. 20 shows light and fluorescent photomicrographs of GLPs confirming that oleamide and leave-in solvent were encapsulated within the GLPs.

Release of Oleamide from GLPs

The release of oleamide encapsulated in GLPs using octanoic acid or undecanoic acid as loading solvent or as leave-in solvent was tested. To perform the release assay, the GLP-oleamide suspensions were diluted with simulated saliva (25% simulated saliva+75% GLP in solvent mixture) with and without the addition of a sample of 75-100 mg fat tissue. The samples were incubated for 1 minute at 37° C., and then the fractions were separated to quantify oleamide in the GLP pellet (unreleased oleamide), oleamide in the supernatant (released) and oleamide in the tissue (released from GLP and absorbed by the tissue). The oleamide absorbed in the tissue was extracted in 100% methanol and the oleamide remaining in GLPs was extracted in 80% ethanol-20% water. The oleamide in all fractions was quantified by HPLC. Table 16 shows the percent of oleamide released from GLPs and remaining in GLPs (in GLP pellet) after one hour after incubation with only saliva or after 5 minutes or 1 hour after incubation with saliva and tissue. Oleamide was released at a low efficiency (2%) from GLPs with simulated saliva and higher efficiency (34-40%) with simulated saliva and tissue. The addition of UA as the leave-in solvent increased OA release from 34% to 46% in 5 minutes and from 40% to 58% with simulated saliva and tissue

TABLE 16
Release of oleamide encapsulated in GLPs
with loading and/or leave-in solvents.

| | % oleamide after | | | | | |
|---|---|---|---|---|---|---|
| | release assay in | | % oleamide after | | | |
| | simulated saliva | | release assay in | | | |
| Sample | (no tissue) | | simulated saliva + tissue | | | |
| (leave-in | 1 hour | | 5 minutes | | 1 hour | |
| solvents) | Released | Pellet | Released | Pellet | Released | Pellet |
| GLP oleamide | 2 | 98 | 34 | 66 | 40 | 60 |
| GLP oleamide | 2 | 98 | 29 | 71 | 49 | 51 |
| (octanoic acid) | | | | | | |
| GLP oleamide | 2 | 98 | 46 | 54 | 58 | 42 |
| (undecanoic | | | | | | |
| acid) | | | | | | |

### LITERATURE CITED

Bakry, A. M.; Abbas, S.; Ali, B.; Majeed, H.; Abouelwafa, M. Y.; Mousa, A.; Liang, L. Microencapsulation of Oils: A Comprehen-462sive Review of Benefits, Techniques, and Applications. Compr. Rev. Food Sci. Food Saf. 2016, 15, 143-182. 463 https://doi.org/10.1111/1541-4337.12179 464
Bard et al. 1988. Geraniol interferes with membrane functions in strains of Candida and Saccharomyces. Lipids 23(6):534-538.
Bhalerao, Y. P.; Wagh S. J. A Review on Thymol Encapsulation and its Controlled Release through Biodegradable Polymer Shells. 465 Int J. Pharm. Sci. 2018, 2, 4522-4532. doi: 10.13040/IJPSR.0975-8232.9(11).4522-32 466
Calo, J. R.; Crandall, P. G.; O'Bryan, C. A.; Ricke S. C. Essential Oils as Antimicrobials in Food Systems—A Review. Food Control. 4602015, 54, 111-119. https://doi.org/10.1016/j.foodcont.2014.12.040 461
Chaumont J. P. and D. Leger, “Campaign Against Allergic Moulds in Dwellings, Inhibitor Properties of Essential Oil Geranium Bourbon, Citronellol, Geraniol and Citral,” Ann. Pharm. Fr. 50 (3): 156-166, 1992.
Dardelle et al. Flavour-Encapsulation and flavour-release performances of a commercial yeast-based delivery system. Food Hydrocoll. 2007, 21. 953-960.
Dimopoulos et al. Cell permeabilization processes for improved encapsulation of oregano essential oil in yeast cells. J. Food Eng. 2021, 294, 110408.
Errenst et al. Encapsulation of limonene in yeast cells using the concentrated powder form technology. J. Supercrit. Fluid 2021, 168, 105076
Friend et al., 1984, J. Med. Chem. 27:261-268.
Gómez, B.; Barba, F. J.; Domínguez, R.; Putnik, P.; Bursać Kovačević, D.; Pateiro, M.; Toldrá, F.; Lorenzo, J. M. Microencapsula-467tion of Antioxidant Compounds through Innovative Technologies and Its Specific Application in Meat Processing. Trends Food 468Sci. Technol. 2018, 82, 135-147. website: doi.org/10.1016/j.tifs.2018.10.006 469.
Hardy et al., 1987 Aliment. Pharmacol. Therap. 1:273-280.
Hooser et al., “Effects of an Insecticidal Dip Containing D-Limonene in the Cat,” J. Am. Vet. Med. Assoc. 189 (8): 905-908, 1986.
Moleyar and Narasimham, 1992. Antibacterial activity of essential oil components. Int J Food Microbiol 16(4).337-342.
Palmisano et al., Advances in Therapy, Vol. 5, No. 4, July/August 1988
Pattnaik, et al. 1997. Antibacterial and antifungal activity of aromatic constituents of essential oils. Microbios 89(358):39-46.
Saifullah, M.; Shishir, M.; Ferdowsi, R.; Rahman, M.; Van Vuong, Q. Micro and Nano Encapsulation, Retention and Controlled 470Release of Flavor and Aroma Compounds: A Critical Review. Trends Food Sci. Technol. 2019, 86, 230-251. 471 website: doi.org/10 1016/j.tifs.2019.02 030
Wyse, J., Luria, G. Trends in intellectual property rights protection for medical cannabis and related products. J Cannabis Res 3, 1 (2021). website: doi.org/10.1186/s42238-020-00057-7.

1. A yeast particle (YP) delivery system comprising:
a YP having a hollow internal space;
a hydrophobic payload substantially encapsulated within the hollow internal space; and an adjuvant.

2. The YP delivery system of claim 1, wherein the YP delivery system further comprises one or more leave-in solvents encapsulated within the hollow internal space of the YP along with the hydrophobic payload

3. The YP delivery system of claim 1, further comprising:
a release agent encapsulated within the hollow internal space of the YP along with the hydrophobic payload; or
a sequestering agent encapsulated within the hollow internal space of the YP along with the payload.

4. (canceled)

5. The YP delivery system of claim 1, wherein the adjuvant comprises a self-emulsifying delivery system (SEDDS) solvent, wherein the SEDDS solvent optionally comprises lecithin, medium chain triglycerides, glycerin, 1,3-propanediol or a mixture thereof.

6. (canceled)

7. The YP delivery system of claim 1, wherein the adjuvant further comprises a drying agent.

8. The YP delivery system of claim 2, wherein the leave-in solvent is selected from the group consisting of glycerin, a fatty acid, a surfactant, undecanoic acid, octanoic acid, glycofurol, and any mixtures thereof.

9. The YP delivery system of claim 3, wherein the release agent is selected from the group consisting of a glycerin, a fatty acid, a surfactant, undecanoic acid, octanoic acid, glycofurol, PLURONIC® 17R4, IGEPAL CO-520, TRITON™ X-100, Brij58, and Brij35 and any mixtures thereof.

10. The YP delivery system of claim 3, wherein the sequestering agent is selected from the group consisting of a fatty acid, octanoic acid, a fatty acid ester mixture, CAPMUL®, NEOBEE® 1053, Nerolidol, a terpene, a surfactant, TRITON™ X100, BRIJ®, PLURONIC®, a glycerin, undecanoic acid, glycofurol, PLURONIC® 17R4, Brij58, and Brij35 and any mixtures thereof.

11. The YP delivery system of claim 1, wherein the hydrophobic payload is selected from the group consisting of tetrahydrocannabinol (THC), cannabigerol (CBG), cannabidiol (CBD), and cannabinol (CBN), and any mixtures thereof, optionally wherein the hydrophobic payload further comprises a second distinct hydrophobic substance.

12. (canceled)

13. A kit comprising the YP delivery system of claim 1.

14. A pharmaceutical composition comprising the YP delivery system of claim 1 and a pharmaceutically acceptable excipient.

15. A method of delivering a hydrophobic payload to a cell comprising contacting a cell with the YP delivery system of claim 1.

16. A method of making a YP delivery system comprising the steps of:
incubating a yeast particle (YP) having a hollow internal space with a hydrophobic payload until the payload becomes substantially enclosed within the hollow internal space; and
embedding the YP in an adjuvant and optionally a drying agent.

17. The method of claim 16, further comprising a step of contacting the YP with
a release agent along with the hydrophobic payload, or
a sequestering agent along with the hydrophobic the hydrophobic payload.

18. (canceled)

19. A method of making a YP delivery system comprising the steps of:
incubating a yeast particle (YP) having a hollow internal space with a hydrophobic payload dissolved in a loading solvent until the payload becomes substantially enclosed within the hollow internal space;
optionally removing the loading solvent; and
embedding the YP in an adjuvant and optionally a drying agent.

20. The method of claim 19, further comprising the step of contacting the YP with
a release agent along with the hydrophobic payload, or
a sequestering agent along with the hydrophobic payload.

21. (canceled)

22. The method of claim 17;
wherein the release agent is selected from the group consisting of a glycerin, a fatty acid, a surfactant, undecanoic acid, octanoic acid, glycofurol, PLURONIC® 17R4, IGEPAL CO-520, TRITON™ X-100, Brij58, and Brij and any mixtures thereof; or
wherein the sequestering agent is selected from the group consisting of a fatty acid, octanoic acid, a fatty acid ester mixture, CAPMUL®, NEOBEE® 1053, Nerolidol, a terpene, a surfactant, TRITON™ X100, BRIJ®, PLURONIC®, a glycerin, a fatty acid, undecanoic acid, glycofurol, PLURONIC® 17R4, Brij58, and Brij35 and any mixtures thereof.

23-24. (canceled)

25. A method of delivering a hydrophobic payload to a subject, comprising administering to a subject the YP delivery system of claim 1.

26. The method of claim 25, wherein the YP delivery system releases the hydrophobic payload within seconds to about ten minutes after administration, optionally wherein the YP delivery system is administered to the subject in the nasal cavity, buccal cavity, via inhalation, topical application, transdermal application, rectal application, vaginal application, otic application, optic application, or any combination thereof.

27-29. (canceled)

30. The method of claim 25, wherein the YP delivery system releases the hydrophobic payload molecule over a period of about 1 hour to about 24 hours after administration, optionally wherein the YP delivery system is administered to the subject in the nasal cavity, buccal cavity, via inhalation, topical application, transdermal application, rectal application, vaginal application, otic application, optic application, or any combination thereof.

31-33. (canceled)
